SEQUENCE LISTING (22 sequences)

SEQ ID NO: 1 - DNA, 2259 bp, Homo sapiens
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 2 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - protein, 497 aa, Homo sapiens
  misc_feature: D409H mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKHTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 4 - protein, 497 aa, Homo sapiens
  misc_feature: P415R mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQRMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 5 - protein, 497 aa, Homo sapiens
  misc_feature: L444P mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDPDAVALMHPDGSAVVVVLNRSSKDVPLTIKDPAVGFLETISPGYSIHTYLWRRQ

SEQ ID NO: 6 - protein, 497 aa, Homo sapiens
  misc_feature: D140H mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPHDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 7 - protein, 497 aa, Homo sapiens
  misc_feature: K157Q mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLQIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - protein, 497 aa, Homo sapiens
  misc_feature: E326K mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGKTHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - protein, 497 aa, Homo sapiens
  misc_feature: D140H+E326K mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPHDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGKTHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 497 aa, Homo sapiens
  misc_feature: G202R mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPRDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - protein, 497 aa, Homo sapiens
  misc_feature: N370S mutant
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITSLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 12 - DNA, 2259 bp, Homo sapiens
  misc_feature: D409H mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaagcacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 13 - DNA, 2259 bp, Homo sapiens
  misc_feature: P415R mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcgcatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 14 - DNA, 2259 bp, Homo sapiens
  misc_feature: L444P mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacccggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 15 - DNA, 2259 bp, Homo sapiens
  misc_feature: D140H mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctcatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 16 - DNA, 2259 bp, Homo sapiens
  misc_feature: K157Q mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctccagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 17 - DNA, 2259 bp, Homo sapiens
  misc_feature: E326K mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctagggaagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 18 - DNA, 2259 bp, Homo sapiens
  misc_feature: D140H, E326K double mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctcatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctagggaagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 19 - DNA, 2259 bp, Homo sapiens
  misc_feature: G202R mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccagagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 20 - DNA, 2259 bp, Homo sapiens
  misc_feature: N370S mutant cDNA
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgagcctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 21 - DNA, 26 bp, Artificial sequence
  single strand DNA oligonucleotide
ctaatgaccctgaattcatggagttt

SEQ ID NO: 22 - DNA, 27 bp, Artificial sequence
  single strand DNA oligonucleotide
gtatctgctcgagcactggcgacgcca

(19) United States
(12) Patent Application Publication
(10) Pub. No.: US 2009/0239807 A1
(43) Pub. Date: Sep. 24, 2009

(54) Methods and kits for diagnosing and/or assessing severity and treating gaucher disease

(76) Inventors: Mia Horowitz, HaSharon (IL); Idit Ron Ronen, Tel Aviv (IL)

Correspondence Address: MARTIN D. MOYNIHAN d/b/a PRTSI, INC., P.O. BOX 16446, ARLINGTON, VA, 22215, US

(21) Appl. No.: 11/597,480

(22) PCT Filed: May 24, 2005
(86) PCT No.: PCT/IL05/00530

§ 371 Date: Nov. 22, 2006

Related U.S. Application Data
(60) Provisional application No. 60/573,335, filed on May 24, 2004.

(51) Int. Cl. A61K 38/07; C12Q 1/34; G01N 33/567

(52) U.S. Cl. 514/18; 435/18; 435/7.21

(57) ABSTRACT

Methods and kits for treating Gaucher disease are provided. The methods are based on using agents capable of inhibiting proteasomal degradation of glucocerebrosidase and/or elevating a level of mis-folded yet active glucocerebrosidase in cell lysosomes. Also provided are methods and kits for diagnosing and/or assessing a severity and determining prognosis of Gaucher disease or other diseases associated with abnormally folded proteins which are retained in the ER.

### FIELD AND BACKGROUND OF THE INVENTION

The present invention relates to methods and kits for diagnosing and/or assessing a severity and/or treating Gaucher disease, and more specifically, to a method of determining the prognosis of an individual carrying two mutated glucocerebrosidase alleles. In addition, the present invention is of a method of diagnosing and/or assessing a severity a disease associated with abnormally folded proteins such as cystic fibrosis, Retinitis Pigmentosa, chronic adult GM2, GM1 gangliosidoses, Morquio B disease and Fabry disease.

Gaucher disease (GD), the most prevalent sphingolipid disorder, is an autosomal recessive disease characterized by the accumulation of glucosylceramide mainly in cells of the reticuloendothelial system. Such accumulation results from impaired activity of the lysosomal enzyme glucocerebrosidase due mainly to mutations in the glucocerebrosidase gene and in some cases to mutations in the gene encoding the glucocerebrosidase activator (saposin C), designated prosaposin (Sandhoff, K, et al., 1995; Christomanou, H., et al., 1989).

Being a very heterogeneous disease, it has been subdivided into three different types on the basis of age of onset, clinical signs and involvement of neurological symptoms. Type 1 (adult type, chronic, non-neuronopathic; MIM# 230800) is the most common form and is characterized by hematological abnormalities with hypersplenism, bone lesions, skin pigmentation, pingueculae (brown spots of Gaucher cells at corneoscleral limbus) and the lack of central nervous system involvement. It is very heterogeneous in its clinical features (Beutler and Grabowski, 1995) and is known as the most prevalent genetic disease among Ashkenazi Jews, with a carrier frequency of 1:17 in the Israeli Ashkenazi Jews (Horowitz et al., 1998); Type 2 (infantile, acute neuronopathic; MIM# 230900) is a rare and lethal form of the disease. It is characterized by early appearance of visceral signs, enlargement of the abdomen from hepatosplenomegaly and central nervous system involvement such as retroflexion of the head, strabismus, dysphagia, choking spells, and hypertonicity. Death occurs usually a few months after birth; Type 3 juvenile, subacute neuronopathic; MIM #321000) is characterized by early onset of visceral impairment (e.g., hepatosplenomegaly) and a later appearance of central nervous system symptoms (Beutler, 1995).

More than 200 Gaucher causing mutations in the glucocerebrosidase gene (GenBank Accession No. D13286) are known to date. Of them, some are associated with the neuronopathic forms of GD, while others are associated with the chronic, adult type (Beutier, E. and Grabowski, G. A., 1995). The mutations include mostly missense point mutations, some frame shift mutations and deletions. Some complex alleles that contain more than one point mutation or point mutations and a deletion were also described (Cormand et al., 2000; Eyal et al., 1990; Grace et al., 1999; Latham et al., 1990; Sinclair et al., 1998). The most prevalent mutations include the N370S mutation (Tsuji et al., 1988) with prevalence of 70% among Ashkenazi patients and 35% among non-Jewish patients; the 84GG mutation (Beutler et al., 1991), which accounts for 12% of the mutated alleles among Ashkenazi Jewish patients; IVS2+1 (He and Grabowski, 1992); L444P (Tsuji et al., 1987), V394L (Theophilus et al., 1989), recTL and recNciI (Eyal et al., 1990). The N370S mutation is associated with a mild form of the disease. The 84GG and the IVS2+1, recNciI, and L444P are associated with neuronopathic manifestation of Gaucher disease. Most Jewish patients are homozygotes for the N370S mutation and most of them exhibit an asymptomatic or a mild form of a disease. Other Jewish patients, who are compound heterozygotes (carry two different mutations), have a more severe disease. Most of these patients have one “neuronopathic” mutation. Most non-Jewish patients are compound heterozygotes with at least one severe, neuronopathic mutation. The percentage of non-Jewish patients suffering from neurological involvement is much higher than that of Jewish patients.

However, some GD patients with identical genotypes exhibit different degrees of disease severity, implicating that a mutation in the glucocerebrosidase gene is required to cause Gaucher disease but other factors play an important role in the manifestation of the disease.

Glucocerebrosidase is a lysosomal membrane-associated glycoprotein which is translated on polyribosomes to a 56 kDa polypeptide. After translocation through the endoplasmic reticulum membrane, accompanied by leader sequence cleavage, the protein is glycosylated on four aspargine residues (Erickson, A. H., et al., 1985). The highly mannosylated sugar moieties are modified while moving through the Golgi network. There, it undergoes further modifications in its sugar moiety, finally being transported to the lysosomes as a 59-63 kDa mature protein by a mannose 6 phosphate receptor independent pathway (Erickson, A. H., et al., 1985; Glickman, J. N. and Komfeld, S., 1993).

In recent reports, few mutations within the glucocerebrosidase gene were suggested to have a trafficking defect. Thus, it was noted that the G202R mutation results in a glucocerebrosidase variant that does not reach lysosomes (Zimmer, K. P. et al., 1999). Addition of sub-inhibitory concentrations of the chemical chaperone N-(n-nonyl)deoxynojirimycin (NN-DNJ) to a fibroblast culture medium derived from a GD patient homozygous to the N370S mutation led to an increase in the activity of the N370S-glucocerebrosidase variant. It was suggested that NN-DNJ led to the proper folding of the N370S mutated enzyme, thus allowing the stabilized enzyme to transit from the endoplasmic reticulum (ER) to the Golgi, enabling proper trafficking to the lysosomes (Sawkar, A. R., et al., 2002). It was also demonstrated that the carbohydrate mimic N-octyl-h-valienamine (NOV), an inhibitor of human glucocerebrosidase (Ogawa, S., et al., 2002), could increase the level of the variant enzyme carrying the F213I mutation and up-regulated cellular enzyme activity in F213I homozygous cells. It was suggested that NOV works as a chemical chaperone to accelerate transport and maturation of F213I carrying glucocerebrosidase (Ogawa, S., et al., 2002; Lin, H., et al., 2004).

It is well documented that mutant proteins are identified as mis-folded by the ER quality control machinery and are retained in the ER. After a certain period of attempts to refold them by the ER chaperons, the misfolded proteins are eliminated from the ER to the cytosol through retrograde transport (Tsai, B, et al., 2002) and are further degraded by the proteasome (Hammond, C. and Helenius, A., 1995; Sitia, R. and Braakman, I., 2003). This whole process is known as the ER associated degradation (ERAD) (Brodsky, J. L. and McCracken, A. A., 1999; Jarosch, E., et al., 2002).

Recent studies suggested that few mutant variants of lysosomal enzymes are retained within the ER. It was shown that in chronic adult forms of GM2 gangliosidoses, resulting from missense mutations in the P-hexosaminidase A, the mutant variants are retained in the ER, resulting in their accelerated degradation (Tropak, M. B., et al., 2004). In the case of β-galactosidase, whose activity is impaired in GM1 gangliosidosis and Morquio B disease, it was shown that some mutant proteins are unstable in the ER/Golgi apparatus and are rapidly degraded without appropriate molecular folding (Zhang, S., et al., 2000). In the case of Fabry disease, caused by reduced α-galactosidase activity, it was shown that, at least in one mutant form (Q279E), the intracellular mutant protein aggregates in the ER and is rapidly degraded (Asano, N., et al., 2000). Very recently, it has been shown that treatment of Fabry fibroblasts, carrying different mutations, with the competitive α-galactosidase inhibitor, 1-deoxygalactonorijimycin (DJG), results in a correction of the lysosomal storage phenotype (Yam, G. H., et al., 2005).

Gaucher disease is diagnosed by biochemical or molecular means. Biochemically, glucocerebrosidase activity is measured in cell lysates from patients, using fluorescent substrates and following their fluorescent derivatives. Molecular diagnosis, executed by PCR amplification of genomic fragments and detection of specific mutations, allows definite characterization of the genotype. However, none of the existing methods allows prediction of disease severity from the genotype.

There is thus a widely recognized need for, and it would be highly advantageous to have, methods of treating and diagnosing and/or assessing a severity Gaucher disease devoid of the above limitations.

### SUMMARY OF THE INVENTION

According to one aspect of the present invention there is provided a method of treating a Gaucher disease in a subject, the method comprising administering to the subject an agent capable of inhibiting proteasomal degradation of glucocerebrosidase thereby treating the Gaucher disease in the subject.

According to another aspect of the present invention there is provided a use of an agent capable of inhibiting proteasomal degradation of glucocerebrosidase for the treatment of Gaucher disease.

According to yet another aspect of the present invention there is provided a use of an agent capable of inhibiting proteasomal degradation of glucocerebrosidase for the manufacture of a medicament identified for the treatment of Gaucher disease.

According to still another aspect of the present invention there is provided a method of treating a Gaucher disease in a subject, the method comprising administering to the subject an agent capable of elevating a level of mis-folded yet active glucocerebrosidase in cell lysosomes, thereby treating the Gaucher disease in the subject.

According to an additional aspect of the present invention there is provided a use of an agent capable of elevating a level of mis-folded yet active glucocerebrosidase in cell lysosomes for the treatment of Gaucher disease.

According to yet an additional aspect of the present invention there is provided a use of an agent capable of elevating a level of mis-folded yet active glucocerebrosidase in cell lysosomes for the manufacture of a medicament identified for the treatment of Gaucher disease.

According to still an additional aspect of the present invention there is provided a method of identifying an agent capable of treating a Gaucher disease, the method comprising: (a) exposing cells expressing an ER-retained glucocerebrosidase to a plurality of molecules; and (b) identifying at least one molecule from the plurality of molecules capable of elevating a level of active glucocerebrosidase in lysosomes of the cells, the at least one molecule being the agent suitable for treating the Gaucher disease.

According to a further aspect of the present invention there is provided a method of diagnosing and/or assessing a severity of Gaucher disease in a subject in need thereof, the method comprising detecting in cells of the subject an ER-retained glucocerebrosidase, wherein a level of the ER-retained glucocerebrosidase is indicative for the severity of Gaucher disease in the subject.

According to yet a further aspect of the present invention there is provided a kit for diagnosing and/or assessing a severity of Gaucher disease in a subject, the kit comprising a packaging material packaging at least one reagent for detecting in cells of the subject a level of an ER-retained glucocerebrosidase thereby diagnosing and/or assessing the severity Gaucher disease in the subject.

According to still a further aspect of the present invention there is provided a method of diagnosing and/or assessing a severity of a disease associated with an abnormally folded protein in a subject the method comprising: detecting a level of an ER-retained form of the protein in cells of the subject, the level being indicative of the severity of the disease associated with the abnormally folded protein.

According to still a further aspect of the present invention there is provided a kit for diagnosing and/or assessing a severity of a disease associated with an abnormal folded protein in a subject, the kit comprising a packaging material packaging at least one reagent for detecting a level of an ER-retained form of the protein in cells of the subject thereby diagnosing and/or assessing a severity of the disease associated with the abnormally folded protein.

According to further features in preferred embodiments of the invention described below, the subject suffers from a type 1, type 2, type 3 or pesudo Gaucher disease.

According to still further features in the described preferred embodiments the agent is a proteasome inhibitor.

According to still further features in the described preferred embodiments the proteasome inhibitor is N-acetyl-leucinyl-leucinyl-norleucinal (ALLN), MG-132, MLN519, benzyloxycarbonyl-isoleucyl-glutamyl(O-tert-butyl)-alanyl-leucinal (PSI) and/or PS-341.

According to still further features in the described preferred embodiments the agent is formulated for systemic administration.

According to still further features in the described preferred embodiments the agent is a small molecule.

According to still further features in the described preferred embodiments the mis-folded yet active glucocerebrosidase includes at least 4 mannose molecules attached to the glucocerebrosidase.

According to still further features in the described preferred embodiments the ER-retained glucocerebrosidase is encoded by a mutated glucocerebrosidase.

According to still further features in the described preferred embodiments the mutated glucocerebrosidase comprises a mutation selected from the group consisting of D409H (SEQ ID NO:3), P415R (SEQ ID NO:4), L444P (SEQ ID NO:5), D140H (SEQ ID NO:6), K157Q (SEQ ID NO:7), E326K (SEQ ID NO:8), D140H+ E326K (SEQ ID NO:9), G202R (SEQ ID NO:10) and N370S (SEQ ID NO:11).

According to still further features in the described preferred embodiments the cells expressing the ER-retained glucocerebrosidase are of a Gaucher disease patient.

According to still further features in the described preferred embodiments the glucocerebrosidase is set forth by SEQ ID NO:2.

According to still further features in the described preferred embodiments the ER-retained glucocerebrosidase includes more than 4 mannose molecules attached to the glucocerebrosidase protein.

According to still further features in the described preferred embodiments detecting is effected by a biochemical analysis and/or a structural analysis.

According to still further features in the described preferred embodiments the biochemical analysis is effected by measuring endo-H sensitivity and/or co-precipitation with an ER-protein.

According to still further features in the described preferred embodiments the ER-protein is calnexin, calreticulin, ERp72, endoplamin (ERp99), ERp29, BIP (GRP78) and GRP94.

According to still further features in the described preferred embodiments the presence of about 15-42% of an endo-H sensitive glucocerebrosidase is indicative of a mild form of Gaucher disease in the subject.

According to still further features in the described preferred embodiments the presence of more than about 60% endo-H sensitive glucocerebrosidase is indicative of a severe form of Gaucher disease in the subject.

According to still further features in the described preferred embodiments detecting is effected by endo-H sensitivity assay.

According to still further features in the described preferred embodiments the protein is a plasma membrane protein or a lysosomal protein.

According to still further features in the described preferred embodiments the plasma membrane protein is selected from the group consisting of CFTR and rhodopsin.

According to still further features in the described preferred embodiments the lysosomal protein is selected from the group consisting of glucocerebrosidase, -hexosaminidase A, and α-galactosidase.

According to still further features in the described preferred embodiments the disease is selected from the group consisting of Gaucher disease, cystic fibrosis, Retinitis Pigmentosa, chronic adult GM2, GM1 gangliosidoses, Morquio B disease and Fabry disease.

According to still further features in the described preferred embodiments the endo-H sensitivity assay is effected using an immunological detection assay.

According to still further features in the described preferred embodiments the endo-H sensitivity assay is effected using a molecule capable of specifically binding a glycoprotein.

The present invention successfully addresses the shortcomings of the presently known configurations by providing methods and kits for diagnosing and/or assessing a severity and/or treating Gaucher disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration depicting a pedigree of a Gaucher disease family. Shown are the genotypes of the different individuals. I, II, and III depict the generation; 1, 2, 3, etc., depict individual number in each generation; +depict normal (wild-type, WT) allele. N.T.=not tested. Note that while one of the brothers (individual II2) is mildly affected, the other brother (individual II4) was severely affected, developed a neurological disease and passed away at the age of 28 from what seemed like Gaucher disease type 3.

FIGS. 2a-e are immunoblots (FIGS. 2a-c) and quantification histograms (FIGS. 2d and e) of Western blot analyses depicting Endoglycosidase H (Endo)-H/Endoglycosidase F (Endo-F) resistance of glucocerebrosidase or hexosaminidase proteins. FIGS. 2a-c—Western blot analyses using antibodies directed against glucocerebrosidase (FIG. 2a), β-hexosaminidase A (FIG. 2c) and erk (FIG. 2b). Cell lysates were prepared from skin fibroblasts, treated with Endo-H (lanes 6-10), Endo-F (lanes 11-15) endoglycosidases (New England Biolabs) or remained untreated (lanes 1-5) and were subjected to Western blot analysis. Lanes 1, 6 and 11—severely affected brother (individual II4 as depicted in FIG. 1); lanes 2, 7 and 12—mildly affected brother (individual II2 as depicted in FIG. 1); lanes 3, 8, and 13—normal foreskin fibroblasts; lanes 4, 9 and 14—the father of the affected brothers (individual I1 as depicted in FIG. 1); lanes 5, 10, 15—the mother of the affected brothers (individual I2 as depicted in FIG. 1). FIG. 2d—a histogram depicting normalized glucocerebrosidase intensity. To normalize the results, intensity of the glucocerebrosidase band at each lane was divided by that of erk. The value obtained for normal glucocerebrosidase was considered 100%. FIG. 2e—a histogram depicting the calculated fraction of Endo-H resistance. The results represent the mean±SEM of 6 independent experiments for the affected brothers and normal cells and 2 independent experiments for the parents. Note that in control cells 99.1% of the glucocerebrosidase is resistant to endo-H degradation. On the other hand, in cells of the severely affected brother only 7% of glucocerebrosidase are endo-H resistant and in cells of the mildly affected brother 29% of glucocerebrosidase are endo-H resistant.

FIGS. 3a-b are an immunoblot (FIG. 3a) and a quantification histogram (FIG. 3b) of Western blot analysis depicting stabilization of glucocerebrosidase by proteasomal inhibitors. Lysates were prepared from skin fibroblasts of two Gaucher brothers: the severely (individual II4) or mildly (individual II2) affected brothers as depicted in FIG. 1, as well as from foreskin fibroblasts (of a normal individual) and were treated with proteasome inhibitors (25 μM ALLN and 10 μM MG-132). Samples of treated and non-treated cells, containing equal amounts of proteins, were electrophoresed through 10% SDS-PAGE and blotted. The blots were reacted with anti-glucocerebrosidase, anti-erk and anti-p53 antibodies. FIG. 3a-Western blot analysis of glucocerebrosidase, erk and p53 antibodies as noted. Lanes 1-3—cells from individual II4, lanes 4-6—cells from individual II2, lanes 7-9—cells from a normal individual. Lanes 1, 4 and 7—untreated cell lysates, lanes 2, 5 and 8—cells lysates treated for 19 hours with proteasome inhibitors; lanes 3, 6, and 9—cell lysates treated for 27 hours with proteasome inhibitors. FIG. 3b—a histogram depicting normalized glucocerebrosidase intensity. To normalize the results, intensity of the glucocerebrosidase band at each lane was divided by that of erk.

FIG. 4 is Western blot analysis depicting recombinant glucocerebrosidase Endo-H sensitivity. HeLa cells were transfected with normal or mutated myc tagged glucocerebrosidase variants: WT (lanes 1, 2), K157Q (lanes 3, 4), D140H (lanes 5, 6), 140/326 (lanes 7, 8), G202R (lanes 9, 10), N370S (lanes 11, 12). Twenty-four hours following transfection lysates were prepared and subjected to Endo-H treatment. Treated (lanes 2, 4, 6, 8, 10 or 12) or non-treated (lanes 1, 3, 5, 7, 9, or 11) lysates were electrophoresed through 10% SDS-PAGE and blotted. Recombinant glucocerebrosidase level was detected by interacting the blot with an anti-myc antibody. Note that while the wild-type (WT) glucocerebrosidase enzyme is resistant to Endo-H treatment, all mutant glucocerebrosidases are sensitive to such treatment.

FIG. 5 is a graph depicting the in vitro activity of glucocerebrosidase in fibroblast cell lysates of GD patients. Samples (subject Nos. correspond to Table 1 of Examples 1 of the Examples section which follows) containing 20 μg of protein were analyzed for glucocerebrosidase activity using 1.5-3 mM of the artificial substrate 4-MUG. The results represent the mean±SEM, as percentage of the activity of normal protein of 3 experiments with 2 repetitions for each one.

FIGS. 6a-d depict glucocerebrosidase levels in normal and GD-derived cells. FIGS. 6a-b are Western Blot analyses depicting the level of glucocerebrosidase (FIG. 6a) and erk (FIG. 6b) proteins. Cell lysates were prepared from either skin fibroblasts of GD patients (subjects Nos. 2-13, numbers correspond to Table 1 of Examples 1 of the Examples section which follows) or foreskin fibroblasts of an unaffected individual (normal; subject No. 1) and aliquots containing the same amount of protein were treated with endo-F and were further subjected to electrophoresis using 10% SDS-PAGE. Western blot analyses were performed using anti-glucocerebrosidase (FIG. 6a) or anti erk (FIG. 6b) antibodies. Note the decreased level of the glucocerebrosidase protein in samples obtained from GD patients (subjects Nos. 7, 3, 12, and 13) as compared with the intensity of the normal protein obtained from subject No. 1. FIG. 6c is a bar graph depicting quantification analysis of the bands obtained in FIGS. 8a and b. The blots were scanned using Image Scan scanner (Amersham Pharmacia Biotech) and the intensity of each band was measured by the image master densitometer 1D prime (Amersham Pharmacia Biotech). To normalize the results, the intensity of glucocerebrosidase measured at each lane was divided by that of erk. The normalized value of glucocerebrosidase obtained for sample No. 1 (unaffected individual) was considered as 100%. FIG. 6d is a bar graph depicting glucocerebrosidase level in GD-derived cells which carry at least one allele with the L444P mutation. Since the anti human glucocerebrosidase monoclonal antibody used in this study does not recognize the L444P mutant protein (Pasmanik-Chor, M., et al., 1997), glucocerebrosidase levels in L444P containing compound heterozygotes (subjects Nos. 4, 7, 8) were compared to those of subject No. 2, who is an individual carrying only one expressed wild type glucocerebrosidase allele (the other allele is null).

FIGS. 7a-d depict endo-H resistance of glucocerebrosidase in GD patients. FIGS. 7a-c—Western blot analyses. Cell lysates were prepared from skin fibroblasts of GD patients (Subjects: 3-13; numbers correspond to Table 1 of Examples 1 of the Examples section which follows) or from normal foreskin fibroblasts (Subject No. 1) and aliquots containing the same amount of protein were either subjected to endo-H digestion (lanes 1, 3, 5, 7, 9, 11, 13, 15, 17, 19, 21, 23) or remained untreated (lanes 2, 4, 6, 8, 10, 12, 14, 16, 18, 20, 22, 24). Following endo-H treatment cell lysates were subjected to Western blot analyses using the anti glucocerebrosidase (GCase; FIG. 7a), anti erk (FIG. 7b) and anti β hexosaminidase A (FIG. 7c) antibodies. Lanes 1-2—subject 1 (WT), lanes 3-4—subject 3 (GD patient—D409H/D409H), lanes 5-6—subject 4 (GD patient—N370S/L444P), lanes 7-8—subject 5 (GD patient—N370S/84GG), lanes 9-10—subject 6 (GD patient—N370S/N370S), lanes 11-12-subject 7 (GD patient—P415R/L444P), lanes 13-14—subject 8 (GD patient—N370S/L444P), lanes 15-16—subject 9 (GD patient—R463C/?), lanes 17-18—subject 13 (GD patient—unknown genotype), lanes 19-20—subject 12 (GD patient -unknown genotype), lanes 21-22—subject 11 (GD patient—N370S/N370S), lanes 23-24—subject 10 (GD patient—N370S/N370S); Subject numbers correspond to Table 1 of Examples 1 of the Examples section which follows. FIG. 7d—Quantification of Western blot analyses shown in FIGS. 7a-c. Band intensity was measured using the Image Scan scanner (Amersham Pharmacia Biotech) and the image master densitometer 1D prime (Amersham Pharmacia Biotech) and Glucocerebrosidase endo-H resistant fraction was calculated. To determine the endo-H resistant fraction the blots were scanned and the intensity of each band was measured. Glucocerebrosidase resistant fraction was calculated by dividing the intensity of the endo-H sensitive fraction (in the endo-H treated samples) by the intensity of the entire amount of glucocerebrosidase in the same lane. The results represent the mean±SEM, as percentage of the endo-H resistant fraction of 4 independent experiments.

FIGS. 8a-u are immunofluorescence images depicting intracellular localization of glucocerebrosidase in GD patients. Cells from normal (WT) or GD patients [subjects Nos. 11, 10, 8, 7, 13, 3 (subject code is shown in Table 1)] were grown on cover-slips, fixed, permeabilized with 0.1% triton X-100 and interacted with anti glucocerebrosidase monoclonal antibody (FIGS. 8a, d, g, j, m, p, s) and an anti calnexin polyclonal antibodies (FIGS. 8b, e, h, k, n, g, t). Detection was performed using the cy-3 conjugated goat anti-mouse antibodies to demonstrate glucocerebrosidase (GCase) localization (red), and using cy-2 conjugated goat anti-rabbit antibodies to demonstrate endogenous calnexin (green). Co-localization was illustrated by merging cy-2 and cy-3 images (Merge; lanes c, f, i, l, o, r, u). The results were visualized using a confocal microscope. Scale bar (10 μm) is the same for all images.

FIGS. 9a-f are immunofluorescence images depicting lysosmal localization of glucocerebrosidase. Normal skin fibroblasts (WT) grown on cover-slips were loaded with Lysotracker (Red; FIGS. 9b and e), fixed with 4% paraformaldehyde, permeabilized with 0.1% triton X-100 and interacted with an anti glucocerebrosidase (GCase) monoclonal antibody (FIGS. 9a and d). Detection of glucocerebrosidase was performed using FITC conjugated goat anti-mouse antibodies (green). Co-localization was illustrated by merging FITC (green) and Lysotracker images (Merge; FIGS. 9c and f). Scale bar: 10 μm.

FIGS. 10a-g depict stabilization of glucocerebrosidase of GD patients in the presence of proteasomal inhibitors. FIGS. 10a-c are Western blot analyses of glucocerebrosidase (GCase; FIG. 10a), p53 (FIG. 10b) and erk (FIG. 10c) of GD-derived fibroblast cell lysates following treatment with proteasome inhibitors. Fibroblast cell lysates from GD patients (subjects 3, 8, 7, 6, 9, 11, 12, 13; subject code as in Table 1) or normal individuals (WT, subject No. 1) were treated for 20 hours with a mixture of proteasome inhibitors (25 μM ALLN and 15 μM MG-132). Aliquots of treated cell lysates (lanes 2, 4, 6, 8, 10, 12, 14, 16, 18) or untreated cells lysates (lanes 1, 3, 5, 7, 9, 11, 13, 15, 17) were subjected to Western blot analysis using anti glucocerebrosidase (FIG. 10a), anti erk (FIG. 10c) and anti p53 (FIG. 10b) antibodies. FIG. 10d-Quantification of Western blot analyses shown in FIGS. 10a-c. Western blot images were scanned using Image Scan scanner (Amersham Pharmacia Biotech) and the intensity of each band was measured by the image master densitometer 1D prime (Amersham Pharmacia Biotech). To normalize the results, glucocerebrosidase intensity at each lane was divided by that of erk and the ratio between treated and untreated protein was calculated. The results represent the mean±SEM, as percentage of the fold increase in protein level due to the treatment of each variant, of 3-6 independent experiments. FIGS. 10e-g—Western blot analyses of glucocerebrosidase (FIG. 10e), p53 (FIG. 10f) and erk (FIG. 10g) of fibroblast cell lysates following treatment with proteasome inhibitors. Fibroblasts cell lysates from a GD patient (subject 7) or an unaffected individual (subject 1) were treated with ALLN (25 μM; lanes 3 and 7 in each of FIGS. 10e-g), MG-132 (15 μM; lanes 2 and 6 in each of FIGS. 10e-g), ALLN and MG-132 (lanes 4 and 8 in each of FIGS. 10e-g), or remained untreated (lanes 1 and 5 in each of FIGS. 10e-g). Aliquots containing equal amounts of protein were subjected to Western blot analyses with anti glucocerebrosidase (FIG. 10e), anti erk (FIG. 10g) or anti p53 (FIG. 10f) antibodies.

FIG. 11 is a Western blot analysis depicting endo-H sensitivity of recombinant glucocerebrosidase variants. Twenty-four hours after transfection of HeLa cells with normal or mutated myc tagged glucocerebrosidase variants (as noted by mutations), cell lysates were prepared and subjected to endo-H treatment. Lysates were electrophoresed through 10% SDS-PAGE and blotted. Recombinant glucocerebrosidase expression was detected by interacting the blot with anti-myc antibody.

FIGS. 12a-o are immuno-fluorescence images depicting localization of glucocerebrosidase and calnexin. HeLa cells, grown on cover-slips, were transfected with normal glucocerebrosidase (WT) or the K157Q, G202R, N370S or D140H mutated forms. Twenty-four hours after transfection cells were fixed and permeabilized with 0.1% triton X-100. Cells were reacted with mouse anti-myc antibody (FIGS. 12a, d, g, j, m) and rabbit anti calnexin antibodies (FIGS. 12b, e, h, k, n). Detection was performed with cy-3 conjugated goat anti-mouse antibodies to demonstrate myc-glucocerebrosidase localization (red), and with cy-2 conjugated goat anti-rabbit antibodies to demonstrate endogenous calnexin (green). Co-localization was illustrated by merging cy-2 and cy-3 images (Merge; FIGS. 12c, f, i, l, o). The results were visualized with a confocal microscope. Scale bar: 10 μm.

FIGS. 13a-l are immuno-fluorescence images depicting localization of glucocerebrosidase and calnexin. HeLa cells, grown on cover-slips, were transfected with normal glucocerebrosidase (WT, shown in FIGS. 12a-c) or the E326K, D140H/E326K, L444P or P415R mutated forms. Twenty-four hours after transfection cells were fixed and permeabilized with 0.1% triton X-100. Cells were reacted with mouse anti-myc antibody (FIGS. 13a, d, g, j) and rabbit anti human calnexin antibodies (FIGS. 13b, e, h, k). Detection was performed with cy-3 conjugated goat anti-mouse antibodies to demonstrate myc-glucocerebrosidase localization (red), and with cy-2 conjugated goat anti-rabbit antibodies to demonstrate endogenous calnexin (green). Co-localization was illustrated by merging cy-2 and cy-3 images (Merge; FIGS. 13c, f, i, l). The results were visualized with a confocal microscope. Scale bar: 10 μm.

FIGS. 14a-i depict the interaction of calnexin with recombinant and endogenous glucocerebrosidase. FIGS. 14a-b are Western blot analyses of anti-myc immunoprecipitation. HEK293 cells were transiently transfected with WT or mutated myc tagged glucocerebrosidase variants and cell lysate were immunopercipitated using an anti myc antibody. The precipitates were electrophorased through 10% SDS-PAGE and blotted, and the corresponding blot was interacted with an anti-myc antibody for the recombinant glucocerebrosidase (FIG. 14b) or with anti-calnexin antibodies (FIG. 14a). Lane 1—WT, lane 2—K157Q, lane 3—D140H, lane 4-E326K, lane 5—D140H/E326K, lane 6—G202R, lane 7—D409H, lane 8—P415R, lane 9—L444P, lane 10—MOCK (transfection mixture), lane 11-non-transfected cells, lane 12—N370S. FIG. 14c-Quantification of Western blot analyses of FIGS. 14a-b depicting normalized clanexin binding to each of the mutant variants. The blots were scanned using Image scan scanner (Amersham Pharmacia Biotech) and the intensity of each band was measured using the image master densitometer 1D prime (Amersham Pharmacia Biotech). To quantify the results, calnexin intensity in each lane was divided by that of glucocerebrosidase. The value obtained for normal glucocerebrosidase (lane 1, WT) was determined as 1. The results represent the mean±SEM of 1-3 independent experiments. FIGS. 14d-i are immunoprecipitation/Western blot analyses depicting the effect of the proteasome inhibitor MG-132 on calnexin interaction with glucocerebrosidase. Cells from normal [subject 1 (WT)] or GD patients [subject 3 (type 3); subject 2 (type 2)] were incubated for 20 hours with MG-132 and their lysates were immunoprecipitated using anti calnexin antibodies (FIGS. 14d and e) or remained without further treatment (FIGS. 14f-i). Immunoprecipitates or whole cell lysates were subjected to electrophoresis through 10% SDS-PAGE, following which the blots were interacted with an anti-myc antibody for recombinant glucocerebrosidase (FIGS. 14e and g), anti-calnexin antibodies (FIGS. 14d and f), an anti-p-53 antibody (FIG. 14h) or an anti-erk antibody (FIG. 14i).

### DESCRIPTION OF THE PREFERRED EMBODIMENTS

The present invention is of methods and kits for diagnosing and/or assessing a severity and treating Gaucher disease. Specifically, the present invention can be used to determine a prognosis of a subject carrying a mutated glucocerebrosidase and to identify agents suitable for treating Gaucher disease.

The principles and operation of a method of diagnosing and/or assessing a severity Gaucher disease according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Gaucher disease (GD) is an autosomal recessive disease characterized by the accumulation of glucosylceramide mainly in cells of the reticuloendothelial system. Such accumulation results mainly from mutations in the glucocerebrosidase gene. GD is a heterogeneous disease consisting of three main types (OMIM #230800, #230900, #321000) and a pseudo disease (OMIM #231005) based on the clinical symptoms and degree of severity. More than 200 mutations have been identified as Gaucher disease-causing-mutations. Some of them are associated with the neuropathic form of the disease (e.g., 84GG and the IVS2+1, recNciI, and L444P), and others are associated with a more mild form of the disease (e.g., N370S). However, in most cases of type 1 GD, identification of the mutations cannot predict the severity and/or prognosis of the individual carrying the mutations.

Glucocerebrosidase is a lysosomal membrane-associated glycoprotein which is translated on polyribosomes, translocated through the endoplasmic reticulum membrane and glycosylated on four aspargine residues. The highly mannosylated sugar moieties are modified while moving through the Golgi network to the lysosomes. Prior art studies demonstrated mutant G202R glucocerebrosidase, obtained from cells of a GD type 2 infant, did not reach the cell lysosomes (Zimmer, K. P. et al., 1999). Although the authors concluded that defective intracellular transport of mutant glucocerebrosidase from the ER to the lysosomes may lead to a more severe clinical phenotype than the residual enzyme activity may indicate, they did not propose using such impaired transport in the diagnosis or determining the severity of GD. Other studies have shown that addition of sub-inhibitory concentrations of the chemical chaperone N-(n-nonyl)deoxynojirimycin (NN-DNJ) can increase the activity of the N370S-glucocerebrosidase variant (Sawkar, A. R., et al., 2002). It was also demonstrated that the carbohydrate mimic N-octyl-h-valienamine (NOV), an inhibitor of human glucocerebrosidase (Ogawa, S., et al., 2002), can increase the level of the variant enzyme carrying the F213I mutation and up-regulate cellular enzyme activity in F213I homozygous cells. It was suggested that NOV works as a chemical chaperone to accelerate transport and maturation of F213I carrying glucocerebrosidase (Ogawa, S., et al., 2002; Lin, H., et al., 2004).

Gaucher disease is currently diagnosed by biochemical or molecular means. Glucocerebrosidase activity is measured in cell lysates using fluorescent substrates. Molecular diagnosis, executed by PCR amplification of genomic fragments and detection of specific mutations, allows definite characterization of the genotype. However, none of the existing methods allows prediction of disease severity and patient's prognosis.

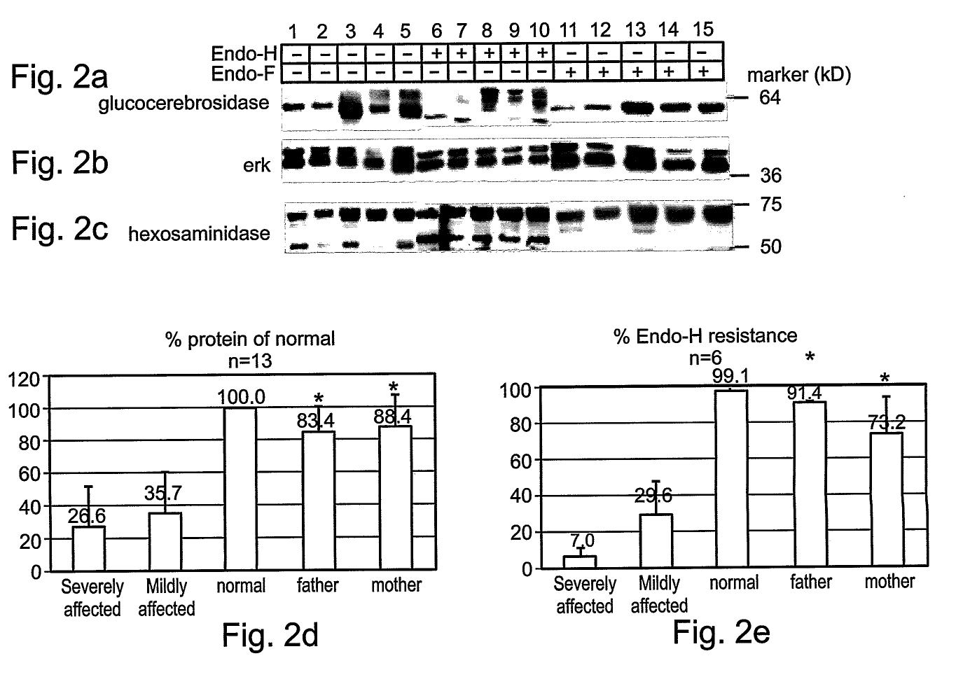

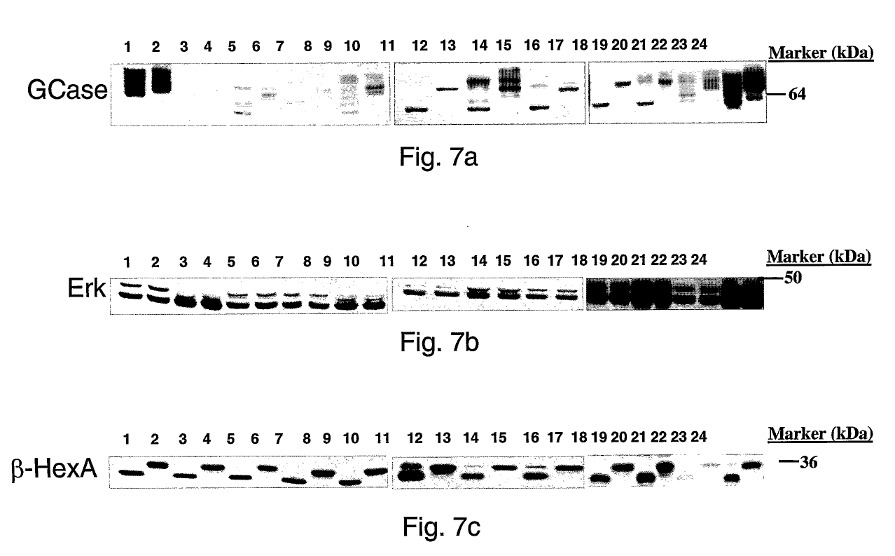

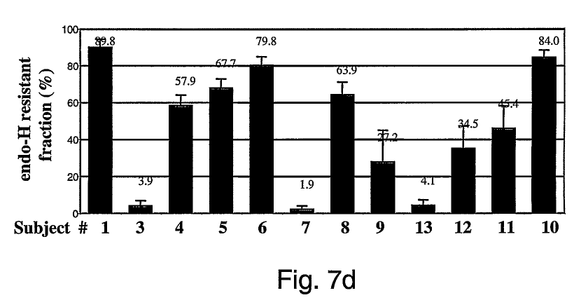

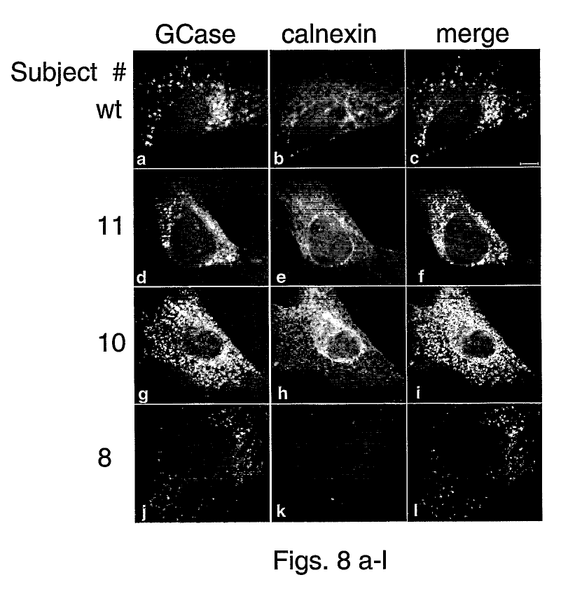

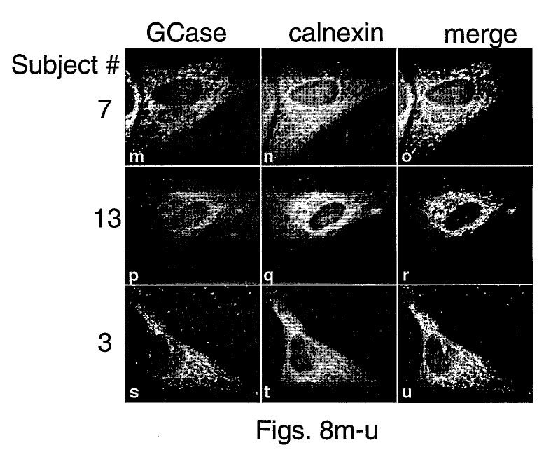

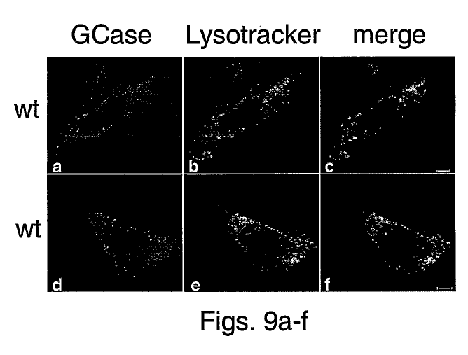

While reducing the present invention to practice, the present inventors have uncovered that the severity and/or prognosis of GD can be predicted by detecting the level of an immature form of glucocerebrosidase. As is shown in FIGS. 2a-e, 4, 7a-d, and 11, and Examples 1-3 of the Examples section which follows, cells of GD patients exhibit immature, highly mannosylated glucocerebrosidase which is sensitive to endo-H digestion. In addition, as is further shown in FIGS. 2e and 7d and in Table 1 of the Examples section which follows, the level of endo-H sensitive glucocerebrosidase was found to correlate with disease severity, even in cases of GD patients sharing the same genotype of glucocerebrosidase mutations. Moreover, the present inventors have further uncovered that the immature glucocerebrosidase is retained in the endoplasmic reticulum (ER) as manifested by its co-precipitation with calnexin, an ER-protein (FIGS. 8a-u, 12a-o, 13a-1, Example 4 of the Examples section which follows). These findings demonstrate, for the first time, that the level of endo-H sensitive glucocerebrosidase can be used to predict the severity of the disease and the patient's prognosis.

Thus, according to one aspect of the present invention there is provided a method of diagnosing and/or assessing a severity Gaucher disease in a subject. The method is effected by detecting in cells of the subject an ER-retained glucocerebrosidase, wherein a level of the ER-retained glucocerebrosidase is indicative of Gaucher disease in the subject.

The phrase “Gaucher disease” encompasses all forms of Gaucher disease and/or pseudo Gaucher disease including, but not limited to, type 1 GD (OMIM NIM #230800), type II GD (OMIM NIM #230900), type III GD (OMIM NIM #321000) and/or pseudo Gaucher disease (NIM# 231005) as described in the background section.

The phrase “diagnosing and/or assessing a severity” as used herein refers to determining presence or absence of a disease, classifying a disease severity or symptom, monitoring disease progression, forecasting an outcome of a disease and/or prospects of recovery. It will be appreciated that the spectrum of mutations causing GD leads to a wide variety of disease severity and those of skills in the arts are capable of distinguishing between a mild or a severe form of the disease.

The term “subject” (or “individual” which is interchangeably used herein) encompasses a human being of any sex who is at risk to develop GD and/or suffers from GD. For example, a subject who is at risk of developing GD is an offspring of two GD-carriers (i.e., individuals who carry a GD disease-causing-mutation). Such an offspring can be a fetus at any embryonic stage, a newbom, a child or an adult. In addition, a subject who is at risk of developing GD can be a subject who carries a GD disease-causing-mutation on both glucocerebrosidase alleles. The phrase “suffers from” refers to an individual exhibiting the clinical signs of GD type 1, 2 or 3 or pseudo GD as described hereinabove. Preferably, the subject whose disease severity is assessed using the method of the present invention is an individual which suffers from type 1 GD.

The phrase “GD disease-causing-mutation” refers to any nucleic acid substitution which is present in cells of the subject and causes GD in a homozygous or compound heterozygous form. Such nucleic acid substitutions can be for example, a missense mutation (i.e., a mutation which results in an amino acid change e.g., N370S, L444P, P415R, R119Q, V394L, D409H, D409V, R463C in glucocerebrosidase), a nonsense mutation (i.e., a mutation which introduces a stop codon in a protein), a frameshift mutation [i.e., a mutation, usually, deletion or insertion of nucleic acids which changes the reading frame of the protein, and may result in an early termination or in a longer amino acid sequence], a readthrough mutation (i.e., a mutation which results in an elongated protein due to a change in a coding frame or a modified stop codon), a promoter mutation (i.e., a mutation in a promoter sequence, usually 5′ to the transcription start site of a gene, which results in up-regulation or down-regulation of a specific gene product), a regulatory mutation (i.e., a mutation in a region upstream or downstream, or within a gene, which affects the expression of the gene product), a deletion [i.e., a mutation which deletes coding or non-coding nucleic acids in a gene sequence, e.g., del72C, 55-bp-del (nucleotides 5879-5933 in a genomic DNA of glucocerebrosidase)], an insertion (i.e., a mutation which inserts coding or non-coding nucleic acids into a gene sequence such as the 84GG in glucocerebrosidase), an inversion (i.e., a mutation which results in an inverted coding or non-coding sequence), a splice mutation [i.e., a mutation which results in abnormal splicing or poor splicing, e.g., IVS 2+1 and IVS DS G-A+1 (which results in a skip of exon 2) in glucocerebrosidase] and a duplication (i.e., a mutation which results in a duplicated coding or non-coding sequence).

As used herein the phrase “ER-retained glucocerebrosidase” refers to a portion of the glucocerebrosidase protein (GenBank Accession No. P04062; glucosylceramide, E.C. 3.2.1.45; GLCM_HUMAN; SEQ ID NO:2) which is immature and thus retained in the ER compartment of cells of the subject. The term “retained” refers to proteins which normally pass through the ER to the Golgi network but are abnormally accumulating in the ER. Such immature proteins are mis-folded and interact with the ER chaperons which attempt to re-fold them. However, following a certain time period, the mis-folded proteins (i.e., the ER-retained proteins) are subject to ubiquitination followed by degradation in the proteasome in a process known as ER-associated degradation (ERAD).

Preferably, the ER-retained glucocerebrosidase is encoded by a mutated glucocerebrosidase (i.e., a glucocerebrosidase gene, mRNA or protein which carries a known or an unknown mutation).

The cells used by the present invention can be any cells which are derived from the subject. Examples include, but are not limited to, blood cells, bone marrow cells, hepatic cells, spleen cells, kidney cells, cardiac cells, skin cells (e.g., epithelial cells, fibroblasts, keratinocytes), lymph node cells, and fetal cells such as amniotic cells, placental cells (e.g., fetal trophoblasts) and/or cord blood cells. Such cells can be obtained using methods known in the art, including, but not limited to, fine needle biopsy, needle biopsy, core needle biopsy and surgical biopsy (e.g., brain or liver biopsy), buccal smear and lavage.

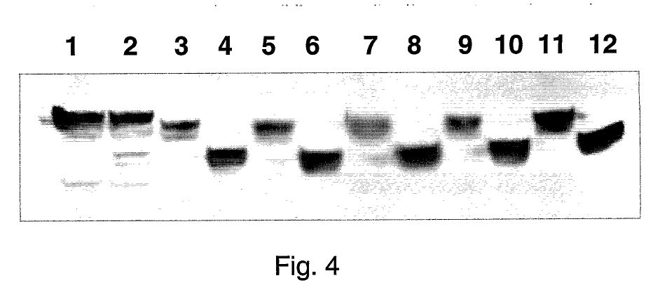

ER-retained proteins can be detected using any structural or biochemical methods which are known in the art for the detection of immature proteins. As is mentioned before and is shown in FIGS. 4, 7a-d, 8a-u, 11, 12a-o, 13a-1, 14-a-i and is described in Examples 1-4 of the Examples section which follows, the present inventors have uncovered that the ER-retained glucocerebrosidase can be detected using an endo-H sensitivity assay, co-precipitation and/or co-localization with an ER-protein or an ER-marker. As used herein, the phrase “ER-protein” refers to any protein which is predominantly localized or present in the ER. Examples include, but are not limited to, calnexin (GenBank Accession No. AAH03552), calreticulin (GenBank Accession No. AAH02500), ERp72 (GenBank Accession No. P38659), endoplamin (ERp99; GenBank Accession No. P08113), ERp29 (GenBank Accession No. P57759), BIP (GRP78; GenBank Accession No. P34935) and GRP94 (Kim PS, and Arvan P, 1998, Endocrine Reviews 19:173-202). The phrase “ER-marker” refers to any molecule which is predominantly present in the ER.

Endo-His a specific endoglycosidase, which can distinguish between highly mannosylated (more than 4 mannose residues) and a mature glycoprotein, which contains the final core of 3 mannose residues, presented in complex oligosaccharides. The removal of two mannose residues to yield the final core of three mannose residues is performed by Golgi mannosidase II in the mid-Golgi.

According to preferred embodiments of the present invention, the ER-retained glucocerebrosidase includes more than 4 mannose molecules which are attached to the glucocerebrosidase protein.

Endo-H sensitivity assay—Briefly, cell lysates are incubated overnight in the presence of endo-H (500 units per 70 μg of total protein in cell lysates) following which the cell lysates are subjected to Western blot analysis using an anti-glucocerebrosidase antibody. To determine the portion of endo-H sensitive glucocerebrosidase, the Western blot images are scanned using a scanner [e.g., Image Scan scanner, Amersham Pharmacia Biotech and an image analysis software (e.g., image master densitometer 1D prime, Amersharn Pharmacia Biotech)] and the intensity of the glucocerebrosidase bands obtained following endo-H digestion is divided by the intensity of glucocerebrosidase obtained in the absence of endo-H.

According to preferred embodiments of the present invention, for the detection of endo-H sensitive glucocerebrosidase, endo-His used at a concentration in a range of 20-1500 units, more preferably, in a range of 50-1000, more preferably, in a range of 300-800, more preferably, about 500 units endo-H per 70 μg of total protein in cell lysates.

It will be appreciated that endo-H sensitive glucocerebrosidase can be also identified using a molecule capable of specifically binding a glycoprotein. Such a molecule can be, for example, a lectin molecule. Various lectins are known in the art and can be used along with the method of the present invention. These include, but are not limited to, legume lectins such as Concanavalin A, Annexins, Ca-dependent (C-type) animal lectins and the like. Thus, such a molecule can be, for example, attached to a solid support for the screening of multiple samples and quantifying the endo-H sensitive portion of glucocerebrosidase in each sample. Briefly, cell lysates are incubated in the presence or absence of endo-H followed by incubation of the cell lysates on ELISA plates containing covalently attached Concanavalin A. Following a pre-determined incubation period (e.g., 15-60 minutes), the plates are washed using e.g., PBS, and are subject to immunostaining using an anti-glucocerebrosidase antibody as described hereinabove. It will be appreciated that the portion of endo-H sensitive glucocerebrosidase can be determined by dividing the glucocerebrosidase-generated ELISA signal in cell lysates incubated in the presence of endo-H to that of cell lysates incubated in the absence of endo-H.

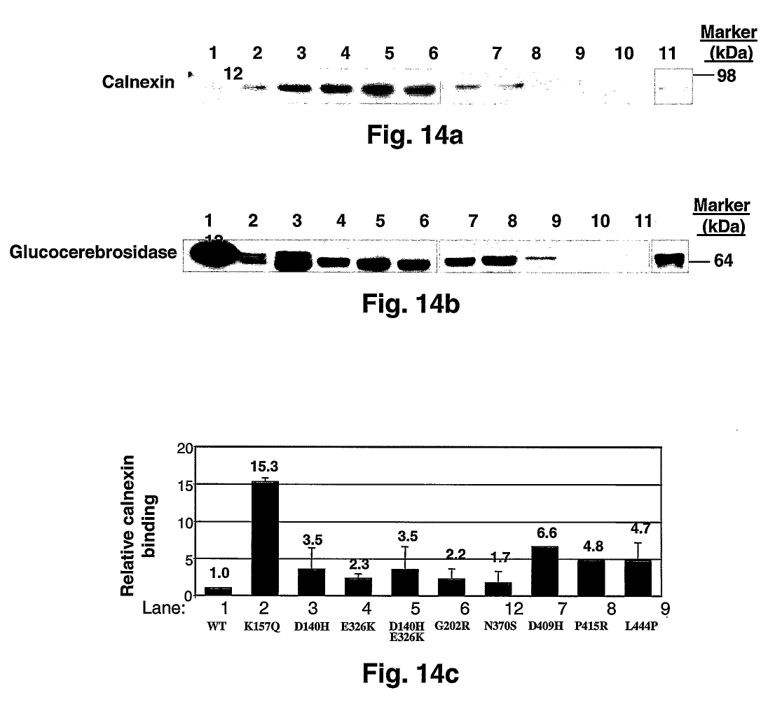

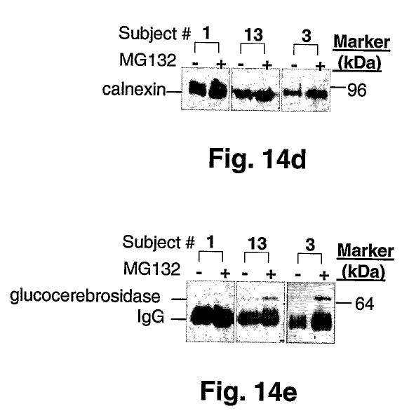

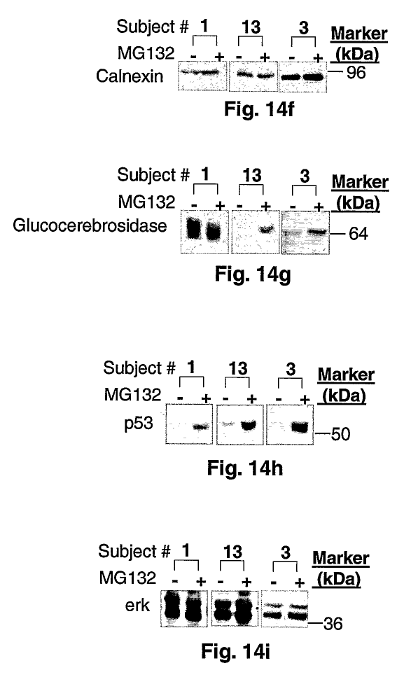

Co-immunoprecipitation of glucocerebrosidase with an ER-protein—Cells derived from a subject (e.g., fibroblast cells of a GD patient) are grown to sub-confluency, washed 0.3 times with ice-cold PBS and then lysed at 4° C. in 1 ml of lysis buffer (10 mM Hepes pH 8, 100 mM NaCl, 1 mM MgCl₂, and 0.5% NP40) containing 10 μg/ml aprotinin, 0.1 mM PMSF, 10 μg/ml leupeptin, 20 mM n-ethyl-maleamide and 10 mM IAA (Sigma-Aldrich, Israel). Cells are incubated with the lysis buffer for 30 minutes on ice following which they are centrifuged for 15 minutes at 10,000 g at 4° C. The supernatants are pre-cleared for 2 hour at 4° C. with protein-A agarose (Roche Diagnostic, Mannheim, Germany). Samples are centrifuged for 1 minute at 15,000 g at 4° C. and the supernatants are incubated overnight at 4° C. in the presence of an anti glucocerebrosidase antibody (e.g., the 8E4 and 2C7 monoclonal anti-glucocerebrosidase antibodies; Pasmanik-Chor M, et al., 1997, Hum Mol. Genet. 6: 887-95) or an antibody directed against an ER-protein. Suitable antibodies for ER-proteins which can be used along with the method of the present invention are the polyclonal anti-calnexin antibodies (e.g., rabbit polyclonal anti-calnexin, SPA-860; Stressgen Biotechnologies, Victoria, BC, Canada) and the ERp29 antibody (AXXORA, LLC San Diego, Calif., Cat # ALX-210-404-R100). For immunoprecipitation, the ER-protein antibodies are preferably immobilized on beads such as the protein A Sepharose (Sigma Aldrich, Israel). Following four washes with 1 ml of lysis buffer containing protease inhibitors, proteins are eluted for 10 minutes at 100° C. using 5× loading buffer are electrophoresed through 10% SDS-PAGE and are blotted with calnexin or ER-protein antibodies (or antibody) essentially as described in the Examples section which follows and is showed in FIGS. 14d-i.

Structural Analysis

Co-localization of glucocerebrosidase with an ER-protein—The localization of glucocerebrosidase in the ER can be detected by immunofluorescence using antibodies directed against glucocerebrosidase and an ER-protein or ER-marker. Subconfluent cells derived from a subject (e.g., fibroblast cells of a GD patient), grown on cover-slips, are washed twice with phosphate buffer saline (PBS), fixed for 5 minutes at 4° C. in methanol, followed by 5 minutes at 4° C. in methanol-acetone (1:1). Following washes, cells are permeabilized for 3 minutes at room temperature (RT) using 0.1% Triton X-100 in PBS and washed 3 times with PBS. Cells are then blocked by incubating for 30 minutes at RT with PBS containing 1% BSA and 20% NGS, and then incubated for 1 hour at RT in the presence of the corresponding primary antibody (e.g., 1:100 dilution for 2C7, 1:200 for rabbit anti-calnexin) in 1% bovine serum albumin (BSA)/PBS. Cells are washed 3 times with PBS and then immunostained for 45 minutes at RT with rabbit-Cy-2 or -mouse-Cy-3 conjugated secondary antibodies (1:200 dilution) in 1% BSA/PBS, following which the cells are washed three times with PBS and mounted using a mounting solution e.g., galvanol on microscopic slides. Co-localization of the glucocerebrosidase and the ER-protein is noted using a fluorescence microscope essentially as described in the Examples section which follows and is showed in FIGS. 8a-u. The degree of co-localization of glucocerebrosidase and the ER-protein can be quantified by measuring the signal of co-localized proteins (e.g., yellow color as shown in FIGS. 8a-u under the “merge” images) as compared with the signal obtained from the ER-protein along (e.g., green color as shown in FIGS. 8a-u under the “calnexin” images). Such a fraction can be compared between GD patients and unaffected individuals and those with skills in the art are capable of correlating specific fractions to disease severity.

It will be appreciated that various other methods can be employed to detect the ER-retained glucocerebrosidase. For example, a structural analysis using an electron microscope can be performed following immunostaining of glucocerebrosidase (using e.g., a monoclonal or polyclonal antibody or antibodies) followed by a secondary, gold-labeled anti mouse antibody (e.g., from E. Y. Laboratories, Inc., San Mateo, Ca.). The localization of the gold labels is indicative of the presence of glucocerebrosidase. Such analysis can be quantified to determine the relative portion of glucocerebrosidase in the ER, Golgi network or the lysosomes and those of skills in the art are capable of correlating a relative portion of ER-retained protein (e.g., glucocerebrosidase) to disease severity.

According to the method of this aspect of the present invention a level of the ER-retained glucocerebrosidase is indicative of the severity of Gaucher disease in the subject. As used herein the phrase “level of the ER-retained glucocerebrosidase” refers to the expression level and/or activity of glucocerebrosidase which is found (retained) in the ER of cells of the subject and which is indicative of GD. It will be appreciated that such level can be calculated as a specific portion out of the total glucocerebrosidase (e.g., a fraction which can be presented in percentage). As is shown in FIGS. 2e and 7d, Table 1 and Example 1 of the Examples section which follows, while in normal, unaffected individuals most of the glucocerebrosidase was endo-H resistant (about 85 to about 99%), the portion of ER-retained glucocerebrosidase in mildly affected GD patients was in the range of 17-42%.

According to one preferred embodiment of the present invention a presence of at least 15% of an endo-H sensitive glucocerebrosidase is indicative of a mild form of Gaucher disease in the subject. Preferably, a presence of at least 20%, more preferably, at least 25%, more preferably, at least 30%, more preferably, at least 35%, more preferably, at least 40%, more preferably, even more preferably, in the range of 17-42% of an endo-H sensitive glucocerebrosidase is indicative of a mild form of Gaucher disease in the subject.

As is shown in FIGS. 2e, 7d, Table 1 and is described in Example 1 of the Examples section which follows, the portion of ER-retained glucocerebrosidase in severely affected individuals was more than 60%.

According to one preferred embodiment of the present invention a presence of more than 55% of an endo-H sensitive glucocerebrosidase is indicative of a severe form of Gaucher disease in the subject. Preferably, a presence of at least 60%, more preferably, at least 65%, more preferably, at least 70%, more preferably, at least 75 more preferably, at least 80%, more preferably, at least 85%, more preferably, at least 90%, more preferably, at least 95%, more preferably, at least 99% of an endo-H sensitive glucocerebrosidase is indicative of a severe form of Gaucher disease in the subject.

It will be appreciated that using the teachings of the present invention, the severity of various other diseases associated with abnormally folded proteins that retain in the ER can be assessed.

Thus, according to another aspect of the present invention there is provided a method of diagnosing and/or assessing a severity a disease associated with an abnormally folded protein in a subject. The method is effected by detecting a level of an ER-retained form of the protein in cells of the subject, the level being indicative of the disease associated with the abnormally folded protein.

As used herein the phrase “abnormally folded protein” refers to any secondary or tertiary structure of a protein which is associated with a presence of a disease.

It will be appreciated that abnormally folded proteins may have a reduced or altered activity due to an altered intracellular localization as described for glucocerebrosidase in the Examples section which follows. Non-limiting examples of diseases which are associated with abnormally folded proteins include, cystic fibrosis [e.g., the AF508 of the CFTR protein (Denning, G. M., et al., 1992; Gelman, M. S., et al., 2003; Xiong, X., et al., 1999), Retinitis Pigmentosa (rhodopsin), chronic adult GM2 gangliosidoses, β-galctosidase [β-hexosaminidase A (Tropak, M. B., et al., 2004, J Biol Chem, 279, 13478-87)], GM1 gangliosidoses, Morquio B disease (Zhang, S., et al., 2000, Biochem J, 348 Pt 3, 621-32), Fabry disease [α-galactosidase (Asano, N., et al., 2000, Eur J Biochem, 267, 4179-86)] and other diseases such as those described in Aridor M and Hannan L A, 2000, Traffic, 1: 836-851; Aridor M and Hannan L A, 2002, Traffic, 3: 781-790; and Kim PS and Arvan P, 1998, Endocrine Reviews 19: 173-202, all of which are fully incorporated herein by references.

According to preferred embodiments of this aspect of the present invention detecting the level of the immature form of the protein is effected by an endo-H sensitivity assay (as exemplified for glucocerebrosidase in Example 1 of the Examples section which follows).

According to other preferred embodiments of the present invention the endo-H sensitivity assay is effected using an immunological detection assay (e.g., Western blot analysis as exemplified for glucocerebrosidase in Example 1 of the Examples section which follows).

Briefly, following endo-H digestion, cell lysates are subject to Western blot analysis using a protein-specific antibody. For example, to detect an immature form of the CFTR protein leading to cystic fibrosis, an antibody directed against an epitope of the CFTR protein is used (e.g., Mills C L, et al., 1992, Biochem. Biophys. Res. Commun. 188: 1146-52). Methods of preparing antibodies are further described hereinunder. Thus, the level of immature protein is calculated in cells of patients and unaffected individuals by quantifying the amount of endo-H sensitive portion of a protein out of the total protein (obtained in the absence of endo-H) and those of skills in the art are capable of correlating specific endo-H sensitive portions with a severity of the disease associated with abnormally folded proteins.

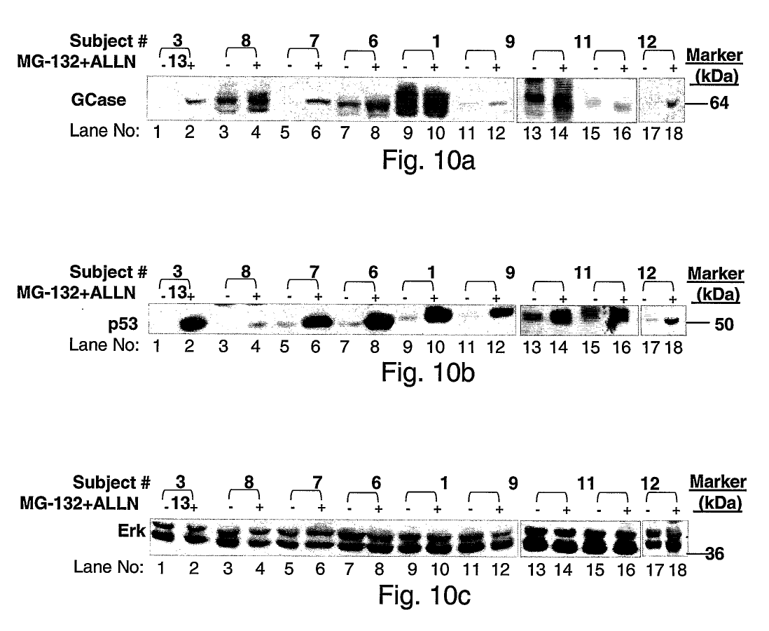

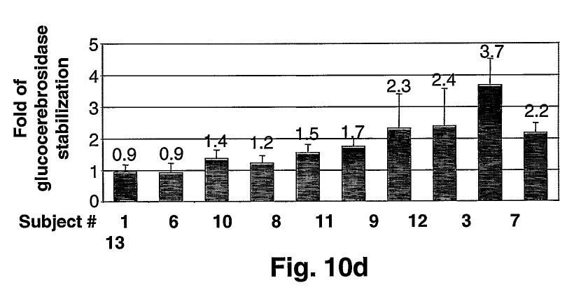

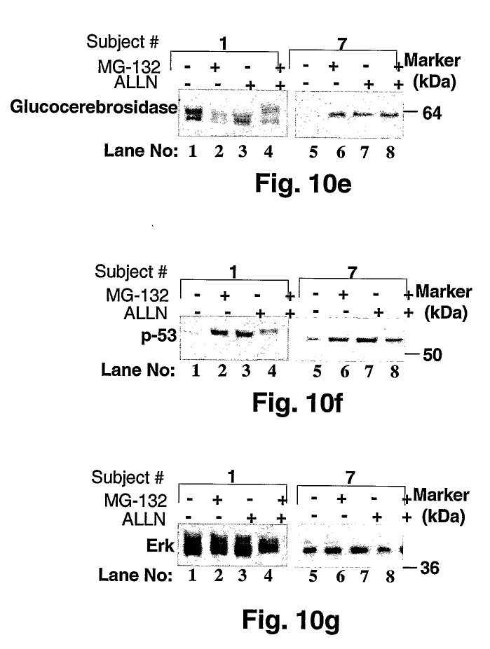

As is shown in FIGS. 10a-g and described in Example 2 of the Examples section which follows, proteasome inhibitors were capable of stabilizing mutated forms of glucocerebrosidase. Thus, in the presence of the proteasome inhibitors MG-132 and ALLN, glucocerebrosidase variants of patients having the neuronopathic form of GD exhibited a 2.2 to 3.8 fold increase in glucocerebrosidase level as compared with glucocerebrosidase variants from patients with type 1 (0.9 to 1.7 increase). These results suggested the use of agents capable of inhibiting degradation via the proteasome for the treatment of GD.

Thus, according to an additional aspect of the present invention, there is provided a method of treating a Gaucher disease in a subject. The method is effected by administering to the subject an agent capable of inhibiting proteasomal degradation of glucocerebrosidase thereby treating the Gaucher disease in the subject.

The term “treating” refers to inhibiting or arresting the development of a disease and/or causing the reduction, remission, or regression of a disease. Those of skill in the art will understand that various methodologies and assays can be used to assess the development of a disease, disorder or condition, and similarly, various methodologies and assays may be used to assess the reduction, remission or regression of a disease.

The term “preventing” refers to keeping a disease, disorder or condition from occurring in a subject who may be at risk for the disease, but has not yet been diagnosed as having the disease.

According to preferred embodiments of this aspect of the present invention the subject suffers from a type 1, type 2, type 3 or pseudo Gaucher disease.

The agent used by the method according to this aspect of the present invention can be any agent capable of inhibiting proteasomal degradation of glucocerebrosidase. Such an agent can be a proteasome inhibitor such as N-acetyl-leucinyl-leucinyl-norleucinal (ALLN), MG-132, MLN519, bortezomib (PS-341) (Luker G D, et al., 2003, Nature Medicine 9: 696-673) and/or benzyloxycarbonyl-isoleucyl-glutamyl(O-tert-butyl)-alanyl-leucinal (PSI). Such proteasome inhibitors can be obtained from any supplier such as Calbiochem (San Diego, Calif., USA) or Millennium Pharmaceuticals.

Additionally or alternatively, such an agent can be any molecule capable of inhibiting the interaction between glucocerebrosidase and components of the ubiquitin machinery (e.g., the E1, E2 or E3, proteasome proteins) which tag mis-folded glucocerebrosidase with a ubiquitin for degradation via the proteasome.

Dosage and modes of administrations of the agent of inhibiting proteasomal degradation of glucocerebrosidase are further described hereinbelow.

As is mentioned before, the severity of GD was associated with a higher portion of endo-H sensitive glucocerebrosidase (i.e., a protein which is retained in the ER) in cells of GD patients. Thus, it will be appreciated that an agent capable of increasing the level of glucocerebrosidase in the lysosomes can be used for treating GD.

Thus, according to yet an additional aspect of the present invention, there is provided a method of treating a Gaucher disease in a subject. The method is effected by administering to the subject an agent capable of elevating a level of mis-folded yet active glucocerebrosidase in cell lysosomes, thereby treating the Gaucher disease in the subject.

As used herein the phrase “mis-folded yet active glucocerebrosidase” refers to a glucocerebrosidase protein (GenBank Accession No. P04062; SEQ ID NO:2) which carries a GD disease-causing-mutation as described hereinabove and is therefore mis-folded (i.e., not properly folded in a secondary and/or tertiary structure). Such a mis-folded glucocerebrosidase variant which accumulates in the ER is potentially active. However, due to its retention in the ER, it is not functional and is further subject to degradation by the ubiquitin machinery.

According to preferred embodiments of the present invention, the mis-folded yet active glucocerebrosidase includes at least 4 mannose molecules attached to the glucocerebrosidase.

The agent according to this aspect of the present invention can be any molecule, including a small molecule, which is capable of elevating a level of mis-folded yet active glucocerebrosidase in cell lysosomes. Examples for such agents and methods of identifying thereof are further described hereinbelow.

The agent of the present invention (i.e., the agent capable of inhibiting proteasome degradation and/or the agent capable of elevating a level of the mis-folded yet active glucocerebrosidase in cell lysosomes) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent accountable for the biological effect, i.e., inhibiting proteasome degradation or elevating a level of the mis-folded yet active glucocerebrosidase.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections, intravenous, inrtaperitoneal, intra-liver, intra-spleen and/or intra-brain.

Preferably, the agent or the pharmaceutical composition containing same is administered by intravenous administration.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g., the agent capable of inhibiting proteasomal degradation of glucocerebrosidase or the agent capable of elevating a level of a mis-folded, yet active glucocerebrosidase in cell lysosomes) effective to prevent, alleviate or ameliorate symptoms of a disorder (i.e., Gaucher disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro, cell culture assays (ex vivo) or animals (in vivo). For example, a dose can be formulated in animal models to achieve a desired concentration or titer. Such information can be used to more accurately determine useful doses in humans.

For example, as is shown in FIGS. 10a-g and is described in Example 2 of the Examples section which follows, in vitro studies utilizing fibroblast cells of GD patients demonstrated that ALLN at a concentration of 25 mM of and/or MG-132 at a concentration of 15 mM are capable of stabilizing mutant variants of glucocerebrosidase.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the level of the active ingredient which is sufficient to inhibit proteasomal degradation of glucocerebrosidase or elevate the level of a mis-folded, yet active glucocerebrosidase in the lysosomes of cells (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that using the teachings of the present invention various other agents can be identified as suitable for treating Gaucher disease.

Thus, according to yet an additional aspect of the present invention there is provided a method of identifying an agent capable of treating a Gaucher disease. The method is effected by: (a) exposing cells expressing an ER-retained glucocerebrosidase to a plurality of molecules; and (b) identifying at least one molecule from the plurality of molecules capable of elevating a level of active glucocerebrosidase in lysosomes of the cells, the at least one molecule being the agent suitable for treating the Gaucher disease.

The “at least one molecule” or the agent described hereinabove which are capable of elevating a level of active glucocerebrosidase in lysosomes of the cells can be for example a peptide, an oligonucleotide, a carbohydrate or any chemical which specifically interacts with a mis-folded yet active glucocerebrosidase and elevates its level in the lysosomes. Such agents (or molecules) can be identified, for example, by screening peptide, oligonucleotide, carbohydrate or any chemical libraries and testing glucocerebrosidase activity in the lysosomes as is further described hereinbelow.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

A peptide library is a combinatorial library, wherein at least some members thereof are peptides having three or more amino acids connected via peptide bonds. In an oligopeptide library, the lengths of the peptides do not exceed 50 amino acids. The peptides may be linear, branched, or cyclic, and may include nonpeptidyl moieties. The amino acids are not limited to the naturally occurring amino acids.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well-within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Carbohydrate libraries can be synthesized employing the “one bead-one molecule” approach, in which the diversity is created by a split-and-pool synthesis or the dynamic combinatorial chemistry (DCC) approach (see for example, Schullek J R, et al., 1997, Anal. Biochem. 246: 20-9; U.S. Pat. Appl. No. 20040146941 to Zhang Biliang et al; Ramstrom O, Lehn J M. Chembiochem. 2000 1: 41-8, which are fully incorporated herein by reference). Such libraries can be screened on cells of the present invention to identify a carbohydrate which specifically interacts with the mis-folded yet active glucocerebrosidase and elevates its level in cell lysosomes.

As is mentioned hereinabove, more than 200 mutations have been identified in GD patients and most of them are likely to form an immature glucocerebrosidase which is retained in the ER. Such mutations can be found in the OMIM database (NCBI) as well as in the Gene Cards database (http:/bioinfo.weizmann.ac.il/cards/index.shtml). It will be appreciated that other, yet unidentified GD-disease-causing-mutations can also lead to the formation of an immature glucocerebrosidase which is retained in the ER and the phrase “mutated glucocerebrosidase” is intended to include all of them a priori.

According to one preferred embodiment of the present invention the mutated glucocerebrosidase can be D409H (SEQ ID NO:3), P415R (SEQ ID NO:4), L444P (SEQ ID NO:5), D140H (SEQ ID NO:6), K157Q (SEQ ID NO:7), E326K (SEQ ID NO:8), D140H+E326K (SEQ ID NO:9), G202R (SEQ ID NO:10), and/or N370S (SEQ ID NO:11).

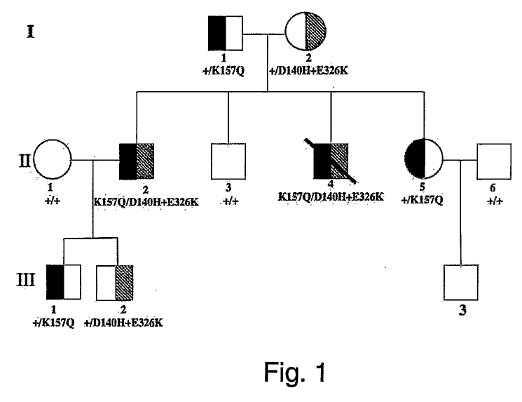

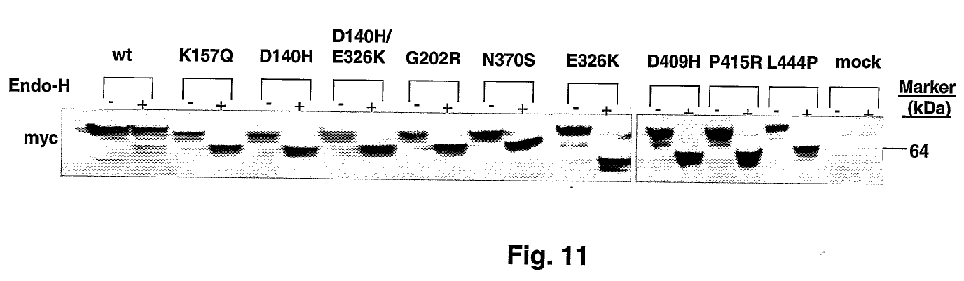

The cells expressing the immature glucocerebrosidase according to this aspect of the present invention can be any cells such as HeLa cells (see FIGS. 4, 11 and Examples 3 and 4 of the Examples section which follows) or HEK293 cells (see FIGS. 14a-i and Example 4 of the Examples section which follows) which are transfected with an expression vector including a polynucleotide encoding a mutated glucocerebrosidase (e.g., SEQ ID NO: 3, 4, or 5) or endogenous cells which are derived from a GD patient such as fibroblast cells and which express a mutated glucocerebrosidase such as the cells described in Example 1 of the Examples section which follows (of the individuals depicted in FIG. 1 and Table 1).

As used herein “identifying at least one molecule . . . capable of elevating a level of active glucocerebrosidase in lysosomes” refers to detecting the presence of an active glucocerebrosidase enzyme in cell lysosomes.

Methods of detecting active glucocerebrosidase in cell lysosomes are known in the art and include the use of fluorescent sphingolipid substrates as described by Madar-Shapiro et al., 1999.

In addition, the enzymatic activity of glucocerebrosidase can be also determined in cell lysates prepared from fractionated cell lysosomes (as described in Asanuma K, et al., 2003, FASEB J. 17: 1165-7) or fractionated ER (Dunkley T P, et al., 2004, Mol. Cell. Proteomics. 3(11): 1128-34). Once obtained, the lysosomal or ER fractionated cell lysates are subjected to glucocerebrosidase activity assay using the appropriate substrates (e.g., 4-MUG) as described under “Materials and Experimental Methods” of the Examples section which follows.

It will be appreciated that increased levels of glucocerebrosidase in the lysosomes can be detected using immunofluorescence with lysosomal specific markers such as lysostracker, essentially as described in the Examples section which follows.

The agents of the present invention which are described hereinabove for detecting the ER-retained glucocerebrosidase or a level of an immature form of a protein may be included in a diagnostic kit/article of manufacture preferably along with appropriate instructions for use and labels indicating FDA approval for use in diagnosing and/or assessing a severity of GD or other diseases associated with an abnormal folded protein.

Such a kit can include, for example, at least one container including at least one of the above described diagnostic agents (e.g., endo-H, anti glucocerebrosidase antibody, a lectin molecule such as Concanavalin A, anti calnexin antibodies, anti CFTR antibody or antibodies) and an imaging reagent packed in another container (e.g., enzymes, secondary antibodies, buffers, chromogenic substrates, fluorogenic material). The kit may also include appropriate buffers and preservatives for improving the shelf-life of the kit.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference); Goldenberg, U.S. Pat. Nos. 4,036,945 and 4,331,647, and references contained therein, which patents are hereby incorporated by reference in their entirety; Porter, R. R. [Biochem. J. 73: 119-126 (1959)]; Inbar et al. [Proc. Nat'l Acad. Sci. USA 69:2659-62 (19720]; Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety; Larrick and Fry [Methods, 2: 106-10 (1991)].

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological, cell biology and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Ma Terials and Experimental Methods

Materials

Antibodies—The following antibodies were used in this study: mouse monoclonal anti-glucocerebrosidase 2C7 (kindly provided by Dr. H. Aerts E. C. Slater Institute for Biochemical Research, University of Amsterdam, the Netherlands); Rabbit polyclonal anti-calnexin (SPA-860; Stressgen Biotechnologies, Victoria, BC, Canada); mouse monoclonal anti-p53 A (DOI, kindly provided by Dr D. Lane, Department of Surgery and Molecular Oncology, University of Dundee, Dundee, United Kingdom.); Rabbit anti-erk (C16 Santa Cruz Biotechnology, Santa Cruz, Calif., USA); rabbit polyclonal anti-hexosaminidase A (kindly provided by Dr R. Gravel, Department of Biochemistry and Molecular Biology, University of Calgary, Calgary, AB, Canada); mouse monoclonal anti-myc (9B11 Cell Signaling Technology, Beverly, Mass., USA). For detection of primary antibodies the following secondary antibodies were used: FITC conjugated goat anti mouse CY-3 conjugated goat anti mouse and CY-2 conjugated goat anti rabbit; horseradish peroxidase conjugated goat anti mouse and goat anti rabbit (Jackson Immuno Research Laboratories, West Grove, Pa., USA).

Proteasome inhibitors—MG-132 and ALLN were purchased from Calbiochem (San Diego, Calif., USA).

Enzymes—Endo-H and -F endo-F were purchased from New England Biolabs (Beverly, Mass., USA). Restriction enzymes were purchased from several companies (as detailed in text) and employed according to manufacturers' recommendations.

The artificial substrate 4-MUG was purchased from Genzyme Corp. (Boston, Mass., USA). NP-40 was purchased from Roche Diagnostic, Mannheim, Germany. Leupeptin was purchased from Sigma Aldrich, Israel.

Experimental Methods

Cell Lines—HeLa (ATCC # CCL-2) and HEK293 (ATCC #CRL-1573) cells were grown in DMEM supplemented with 10% fetal calf serum (FCS). All cells were grown at 37° C. in the presence of 5% CO₂. Human primary skin fibroblasts and foreskin fibroblasts were grown in DMEM supplemented with 20% FCS.

Plasmid construction—Glucocerebrosidase, containing its 39 amino acid residue signal was cloned into the EcoRI and XhoI sites of pcDNA4 myc-his-plasmid B MCS (Invitrogen Life technologies Co. Carlsbad, Calif., USA) using the GC-EcoRI-F (SEQ ID NO:21, 5′-CTAATGACCCTGAATTCATGGAGTTT) and the GC-XhoI-R (SEQ ID NO:22, 5′-GTATCTGCTCGAGCACTGGCGACGCCA) primers which include the restriction sites of EcoRI and XhoI, respectively. The GC-XhoI-R was designed such that the resulting TGA (stop codon of glucocerebrosidase cDNA) is modified from TGATGGAG (nucleotide coordinates 1714-1721 as set forth in SEQ ID NO:1) to TGCTCGAG. The glucocerebrosidase cDNA and plasmids were digested with EcoRI and XhoI and further ligated into the +B MCS of the pcDNA4myc-His. Following ligation, the 5′-end of the cloned glucocerebrosidase cDNA was nucleotide 106 as set forth in SEQ ID NO:1 (GenBank Accession No. D13286). To create variant forms with specific mutations, in vitro site directed mutagensis was performed, using the Quick Change site directed mutagenesis kit (Stratagene Life-Technologies Co., Austin, TA, USA). Amplified products were digested with DpnI to remove contaminating parental plasmid DNA and subsequently transformed into DH5-α Competent E. coli cells. The introduced mutations were: D140H (amino acid sequence—SEQ ID NO:6; nucleic acid sequence—SEQ ID NO:15; G640C;), K157Q (amino acid sequence—SEQ ID NO:7; nucleic acid sequence—SEQ ID NO:16; A691C), E326K (amino acid sequence—SEQ ID NO:8; nucleic acid sequence—SEQ ID NO:17; G1198A), D140H+E326K (amino acid sequence—SEQ ID NO:9; nucleic acid sequence—SEQ ID NO:18; G640C+G1198A), G202R (amino acid sequence—SEQ ID NO:10; nucleic acid sequence—SEQ ID NO:19; G826A), N370S (amino acid sequence—SEQ ID NO:11; nucleic acid sequence—SEQ ID NO:20; A1331G), D409H (amino acid sequence—SEQ ID NO:3; nucleic acid sequence—SEQ ID NO:12; G1446C), P415R (amino acid sequence—SEQ ID NO:4; nucleic acid sequence—SEQ ID NO:13; C1466G) and L444P (amino acid sequence—SEQ ID NO:5; nucleic acid sequence—SEQ ID NO:14; T1553C). Mutations were confirmed by DNA sequencing. Mutation numbering corresponds to the wild type cDNA of glucocerebrosidase as set forth in SEQ ID NO:1. The protein variants depicted in SEQ ID NOs: 3, 4, 5, 6, 7, 8, 9, 10, 11, correspond to the accepted terminology of the GD mutations (OMIM, NCBI) which refer to the processed glucocerebrosidase lacking the 39 amino acid residues of the leader sequence.

Endo-H and endo-F treatment—Samples of cell lysates, containing 70 μg of total protein, were subjected to an overnight treatment with endo-H or endo-F according to the manufacturer's instructions.

Proteasomal inhibition—Subconfluent human skin fibroblasts were grown on 9 mm plates in the presence or absence of 25 mM ALLN and 15 mM MG-132. Twenty hours later, protein lysates were prepared and aliquots containing the same amount of protein, as determined by the Bradford technique (Bradford, M. M., et al., 1976), were subjected to Western blot analysis.

SDS-PAGE and Western Blotting—Cell monolayers were washed 3 times with ice-cold PBS and lysed at 4° C. in 500 μl of lysis buffer (10 mM Hepes pH 8.0, 100 mM NaCl, 1 mM MgCl₂, and 1% TritonX 100) containing 10 μg/ml aprotinin, 0.1 mM PMSF and 10 μg/ml leupeptin (Sigma-Aldrich, Israel). Lysates were incubated on ice for 30 minutes and centrifuged at 10,000 g for 15 minutes at 4° C. Aliquots containing the same amount of protein were electrophoresed through 10% SDS-PAGE and electroblotted onto a nitrocellulose membrane (Schleicher & Schuell BioSience, Keene, N.H., USA). Membranes were blocked with 5% skim milk and 0.1% Tween 20 in TBS for 1 hour at RT, and incubated with the primary antibody for 1 hour at RT. The membranes were then washed 3 times in 0.1% Tween-20 in TBS and incubated with the appropriate secondary antibody for 1 hour at RT. After washing, membranes were reacted with ECL detection reagents (Santa Cruz Biotechnology, Inc. Santa Cruz, Calif.) and analyzed by luminescent image analyzer (Kodak X-OMAT 2000 Processor, Kodak Rochester, N.Y., USA).

Transfections—Transfection was performed using Fugene transfection reagent (Roche Diagnostic, Mannheim, Germany) according to the manufacturer's instructions.

Immunoprecipitation—HeLa and HEK293 cells were transiently transfected with plasmid expressing wild type (WT) or mutated myc tagged glucocerebrosidase. Forty-eight hours after transfection, the cells were washed 3 times with ice-cold PBS and then lysed at 4° C. in 1 ml of lysis buffer (10 mM Hepes pH 8, 100 mM NaCl, 1 mM MgCl₂, and 0.5% NP40) containing 10 μg/ml aprotinin, 0.1 mM PMSF, 10 μg/ml leupeptin, 20 mM n-ethyl-maleamide and 10 mM IAA (Sigma-Aldrich, Israel). Cells were incubated with the lysis buffer for 30 minutes on ice following which they were centrifuged for 15 minutes at 10,000 g at 4° C. The supernatants were then pre-cleared for 2 hours at 4° C. with protein-A agarose (Roche Diagnostic, Mannheim, Germany). Samples were centrifuged for 1 minute at 15,000 g at 4° C. and the supernatants were incubated overnight at 4° C. in the presence of the monoclonal anti-myc or the polyclonal anti-calnexin antibodies immobilized on protein A Sepharose (Sigma Aldrich, Israel). Following four washes with 1 ml of lysis buffer containing protease inhibitors, proteins were eluted for 10 minutes at 100° C. using 5× loading buffer, electrophoresed through 10% SDS-PAGE and blotted. The corresponding blot was interacted with the appropriate antibodies.

Immunostaining, immunocytochemistry and confocal laser scanning microscopy—Subconfluent cells, grown on cover-slips, were washed twice with PBS, fixed for 5 minutes at 4° C. in methanol, followed by 5 minutes at 4° C. in methanol-acetone (1:1). Following washes, cells were permeabilized for 3 minutes at room temperature (RT) using 0.1% Triton X-100 in PBS and washed 3 times with PBS. For immunostaining, the cells were blocked by incubating for 30 minutes at RT with PBS containing 1% BSA and 20% NGS, and then incubated for 1 hour at RT in the presence of the corresponding primary antibody (1:100 dilution for 2C7, 1:200 for rabbit anti-calnexin and 1:5000 for anti-myc) in 1% BSA/PBS. Cells were washed 3 times with PBS and then immunostained for 45 minutes at RT with rabbit-Cy-2 or -mouse-Cy-3 conjugated secondary antibodies (1:200 dilution) in 1% BSA/PBS. Following three washes with PBS, the cover-slips were mounted with galvanol. For lysostracker colocalization (immunohistochemistry), cells were loaded for 1 hour with 25 nM of lysotracker (Lysotracker Red DND-99 Molecular probes, Eugene, Oreg., USA) at 37° C., were fixed 15 minutes in 4% parformaldehyde and further treated (i.e., permeabilized and washed) as described for immunostaining hereinabove. Cells were then immunostained with an anti glucocerebrosidase antibody, washed and further incubated with FITC conjugated goat anti mouse antibodies. Cells were observed and analyzed with a LSM 510 confocal laser scanning microscope (Carel Zeiss, Germany).

Enzymatic activity—Confluent primary skin fibroblasts were washed twice with PBS, collected with a rubber policeman in 1 ml sterile water and frozen in aliquots at −80° C. Twenty μg of total cell lysates were assayed for acid β-glucocerebrosidase activity in 0.2 ml of 100 mM potassium phosphate buffer, pH 4.5, containing 0.15% Triton X-100 (v/v, Sigma) and 0.125% taurocholate (w/v, Calbiochem, San Diego, Calif., USA) in the presence of 1.5 mM 4-MUG, for 60 minutes at 37° C. The reaction was stopped by addition of 1 ml 0.1 M glycine, 0.1 M NaOH pH 10. The amount of 4-MU was quantified using Perkin Elmer Luminescence Spectrometer LS 50 (excitation length: 340 nm; emission: 448 nm).

### Example 1

### Gaucher Disease Patients Exhibit Immature Glucocerebrosidase which is Endo-H Sensitive

The present inventors have investigated a non-Jewish family with two Gaucher affected brothers, carrying the same three mutations. One allele, that derived from the father, carried the K157Q mutation, while the other allele, deriving from the mother, had two base pair changes resulting in D140H and E326K (Eyal et al., 1991), as depicted in FIG. 1. While one of the brothers is mildly affected (II2), the other brother (II4) was severely affected, developed a neurological disease and eventually passed away at the age of 28 from what seemed like Gaucher disease type 3. To understand the molecular basis underlying the difference in disease severity between GD patients carrying the same mutations, several lines of research were pursued, as follows.

Experimental Results

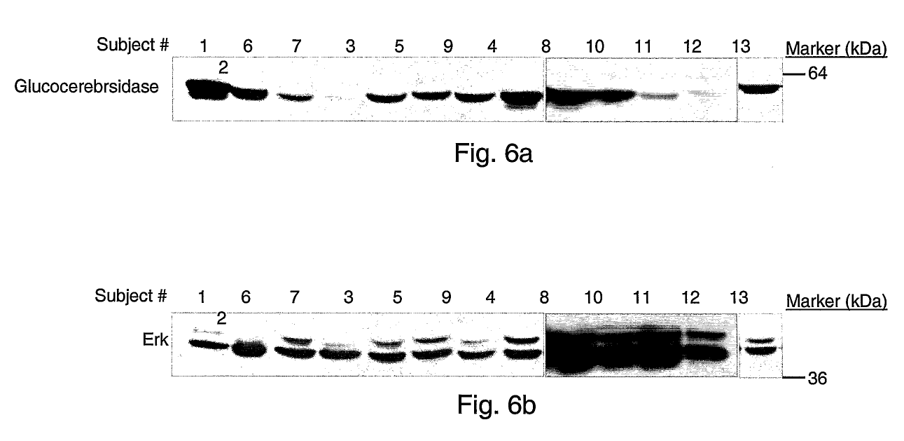

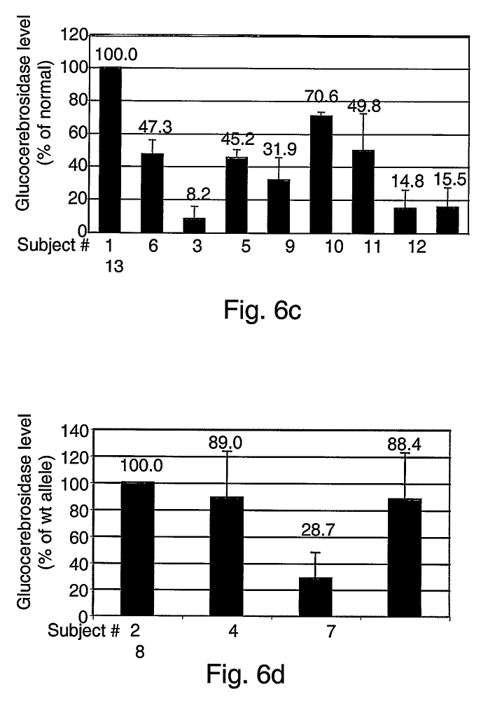

Decreased glucocerebrosidase levels in GD fibroblasts after endo-F digestion—Fibroblast cells derived from various GD patients were tested for the level of glucocerebrosidase after endo-F digestion. Endo-F is an endoglycosidase that removes all aspargine-linked glycosylations from a glycoprotein (Plummer, T. H., et al., 1984; Trimble, R. B. and Tarentino, A. L., 1991; Maley, F., et al., 1989), thus resulting in one glucocerebrosidase isoform which can be readily detected by Western blot analysis. As presented in FIGS. 6a-d and Table 1, hereinbelow, most patients exhibited decrease in glucocerebrosidase level compared to normal cells with some correlation to disease severity. Notably, there was a significant decrease in the amount of glucocerebrosidase in skin fibroblasts derived from type 2 and type 3 patients. To ensure that the decrease in glucocerebrosidase level did not reflect a general decrease in level of lysosomal enzymes, β-hexosaminidase A levels were tested. The results demonstrated no difference in β-hexosaminidase A level (data not shown), indicating that the decrease in protein level is specific to glucocerebrosidase.

TABLE 1
Correlation between glucocerebrosidase level, endo-H
resistance and the clinical manifestations in GD patients

| | | | GCase | | | | | |
|---|---|---|---|---|---|---|---|---|
| | | Disease | levels | | Genotype- | | | Genotype- |
| Subject | | type | (% of | | phenotype | % endo-H | | phenotype |
| no. | Genotype | (severity) | normal) | std | correlation | resistant | std | correlation |
| 1 | WT | normal- | 100 | 0 | normal | 89.8 | 4.6 | + |
| 2 | WT/84GG | carrier | ** | | carrier | 91.6 | | + |
| 3 | D409H/D409H | 3 | 8.2 | 7.3 | + | 3.9 | 2.8 | + |
| 4 | N370S/L444P | 1(mild) | * 99.3 | 33.8 | + | 57.9 | 5.9 | + |
| 5 | N370S/84GG | 1(mild) | 62.16 | 33.4 | + | 67.7 | 4.9 | + |
| 6 | N370S/N370S | 1(mild) | 47.3 | 8.5 | − | 79.8 | 4.9 | + |
| 7 | P415R/L444P | 2 | * 34.8 | 18.7 | + | 1.9 | 1.9 | + |
| 8 | N370S/L444P | 1(mild) | * 69.2 | 42.2 | + | 63.9 | 6.9 | + |
| 9 | R463C/? | 1(severe) | 31.9 | 13.3 | + | 27.2 | 17.4 | + |
| 10 | N370S/N370S | 1(mild) | 70.63 | 2.3 | + | 84.9 | 4.4 | + |
| 11 | N370S/N370S | 1(severe) | 49.75 | 22.3 | + | 43.4 | 12.5 | + |
| 12 | unknown | 3 | 14.8 | 11.2 | + | 34.5 | 12.4 | + |
| 13 | unknown | 2 | 15.53 | 11.8 | + | 4.1 | 3.6 | + |

Table 1: The correlation between glucocerebrosidase (GCase) levels, endo-H resistance, the clinical manifestations and the genotyope-phenotype correlation are presented. Patients with unknown mutation were excluded for the existence of the N370S, L444P, P415R, 84GG, IVS2, D409H, recTL and recNciI mutations.
Std—standard deviation.
** cells from this individual were used as a control for compound heterozygotes with one undetected allele (L444P) and were not compared to wild type (WT) cells.
* results obtained for these individuals were compared to those obtained for subject No. 2.

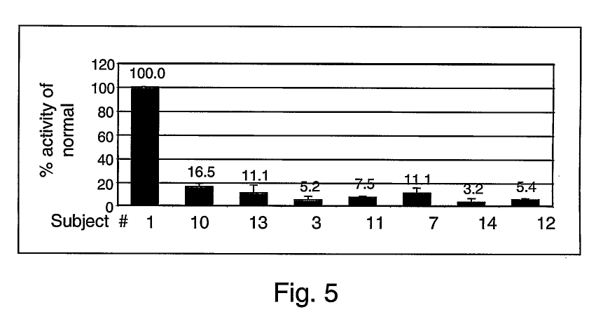

No correlation between endogenous glucocerebrosidase activity and GD severity—The endogenous glucocerebrosidase activity of various GD patients was tested in vitro by subjecting lysates of primary skin fibroblasts derived from different GD patients to the artificial substrate 4-MUG. As shown in FIG. 5, all samples demonstrated low glucocerebrosidase activity, of about 3.2-16.5% of normal (i.e., fibroblasts from a healthy individual). There was no correlation between glucocerebrosidase activity and disease severity (Table 1, hereinabove), indicating that the in-vitro activity of mutated glucocerebrosidase variants cannot be used to predict GD severity.

Thus, the variability in disease severity among GD patients having the same genotype (e.g., the two affected brothers, II2 and II4 as depicted in FIG. 1) can not be explained by glucocerebrosidase activity.

The present inventors hypothesized that the variability in GD phenotypes can result from variability in glucocerebrosidase transport into the lysosomes.

To test this hypothesis, fibroblast cell lysates of control (WT) or GD patients were subjected to degradation by endoglycosidase H (Endo-H) or endoglycosidase F (PNGase-F; Endo-F). Endo-His a specific endoglycosidase, which can distinguish between highly mannosylated (more than 4 mannose residues) and a mature glycoprotein, which contains the final core of 3 mannose residues, presented in a complex oligosaccharides. The removal of two mannose residues to yield the final core of three mannose residues is performed by Golgi mannosidase II in the mid-Golgi. Therefore Endo-H can distinguish between unprocessed protein that did not reach the mid-Golgi apparatus and folded, processed protein that already passed the mid Golgi apparatus. Endo-F removes all aspargine-linked glycosylations and was used to confirm that the changes in protein migration result from different protein glycosylations and not from the changes in amino-acid sequence.

Cells derived from the two Gaucher disease brothers exhibit different degrees of Endo-H resistance—Fibroblast cell lysates from both brothers (individuals II2 and II4 of FIG. 1) were incubated with Endo-H or Endo-F enzymes. As shown in FIGS. 2a-e, there was a significant difference in the Endo-H cleavage pattern of the two affected brothers as compared with each other (lanes 6 and 7 in FIG. 2a) as well as in comparison with the Endo-H cleavage pattern obtained in cells derived from control fibroblasts (lane 8, FIG. 2a) or from the carrier parents (lanes 9 and 10, FIG. 2a). In control cells (WT), 99.1% of glucocerebrosidase was Endo-H resistance (FIG. 2e) demonstrating that most of the protein was processed and had already reached the mid-Golgi apparatus (probably mature and lysosomal). On the other hand, 29% of the glucocerebrosidase in cells of the mildly affected brother (individual II2) and 7% of the glucocerebrosidase in cells of the severely affected one (individual II4) were Endo-H resistance (FIG. 2e), suggesting that most of the glucocerebrosidase in these cells (of the GD patients, individuals II2 and II4) was unprocessed and did not reach the mid Golgi apparatus. The significant difference in Endo-H resistance between the brothers may shed a light on the difference in clinical manifestations of the disease.

On the other hand, the Endo-F cleavage pattern of glucocerebrosidase was the same in both affected brothers (individuals II2 and II4 as depicted in FIG. 1) as in control cells (FIG. 2a), arguing that the difference in Endo-H cleavage pattern results from the difference in glycosylation.

Glucocerebrosidase stability and Endo-H sensitivity was also tested in cells from the parents of the two affected brothers. The results exemplify a slight reduction in glucocerebrosidase level (83-88% of normal; FIG. 2d) in both parents, as well as in Endo-H sensitivity (91% in the father's cells and 71% in the mother's cells compared to 99.1% in normal cells; FIG. 2e).

Endo-H sensitivity of glucocerebrosidase derived from additional GD patients—As is shown in FIGS. 7a-d, in control cells about 90% of glucocerebrosidase was endo-H resistant, indicating that most of the protein was processed and passed already the mid-Golgi apparatus (probably mature lysosomal). On the other hand, 58-83% of glucocerebrosidase in cells of mildly affected type 1 patients and only 1.8-4% of the enzyme in cells of neuronopathic patients were endo-H resistant, suggesting that a significant fraction of glucocerebrosidase in these cells is unprocessed, did not reach the mid Golgi apparatus and therefore was not lysosomal. Furthermore, a significant difference in endo-H cleavage pattern was observed between patients with the same genotype but with different disease severity. There was a direct correlation between the levels of endo-H sensitive fractions and the disease severity presented by the patients. Patients homozygous for the N370S mutation with a mild form of type 1 disease (subjects 6 and 10) demonstrated 80-84% endo-H resistance, whereas N370S homozygous patients with severe type 1 disease (subjects 9 and 11) exhibited only 27-45% endo-H resistant glucocerebrosidase. In addition, compound heterozygotes with the genotype N370S/L444P (patients 4 and 8), in whom only the N370S protein could be detected (Pasmanik-Chor, M., et al., 1997), or N370S/84GG, in whom one allele is not expresses (subject 5), presented 58-68% of endo-H resistant glucocerebrosidase (FIG. 7d).

To verify that the difference in Endo-H cleavage pattern shown hereinabove is not due to a defect in sorting or processing of lysosomal proteins, Endo-H and Endo-F sensitivity of another lysosomal protein β Hexosaminidase A was tested. Hexosaminidase A is a lysosomal protein responsible for the degradation of GM2 gangliosides by hydrolysis of its terminal N-acetyl-galactoseamine residue. It is composed of two α and two β subunits (Gravel et al., 1995). The results demonstrated no difference in endo-H cleavage pattern of β⁻ hexosaminidase A between control and GD cells [FIG. 7c and additional unshown data of the GD brothers (individuals II2 and II4 of FIG. 1)], indicating that the difference in endo-H sensitivity is specific to glucocerebrosidase and there is no general defect in sorting of lysosomal proteins or their processing in GD patients.

These results demonstrate, for the first time, a correlation between the severity of Gaucher disease symptoms and the glycosylation state of glucocerebrosidase and suggest the use of endo-H sensitivity for diagnosing and/or assessing a severity of a GD patient.

### Example 2

### Endogenous Immature Glucocerebrosidases are Subject to ER Associated Degradation (ERAD)

One possible explanation to the presence of immature glucocerebrosidases in the affected GD patients is that such proteins retain in the ER and undergo ER associated degradation (ERAD). In this process mutated proteins are identified as mis-folded and are recognized by ER chaperones which attempt to refold them. After a certain period, the unfolded proteins are tagged by ubiquitin and eliminated from the ER to the cytosol through retrograde transport and get degraded by the proteasome (Bonifacino and Weissman, 1998; Tsai and Rapoport, 2002). If this is the case for glucocerebrosidase of GD patients then the use of proteasomal inhibitors such as MG 132 and ALLN should stabilize the mis-folded glucocerebrosidase.

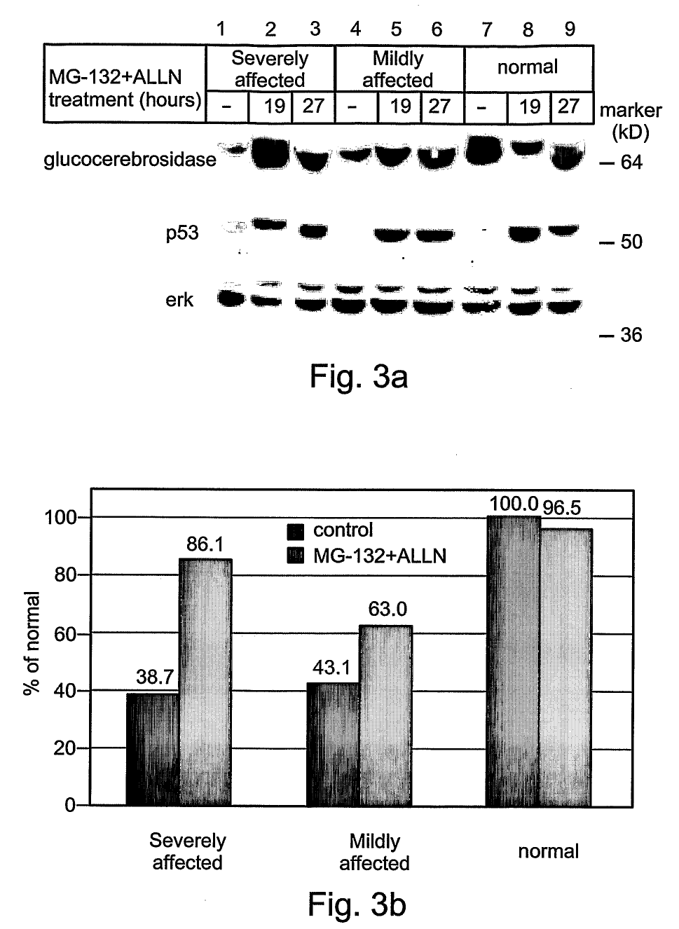

Proteasome inhibitors stabilize mutant glucocerebrosidase variants—To this end, cells from both affected GD brothers (individuals II2 and II4 as depicted in FIG. 1), as well as normal cells, were subjected to 19 or 27 hours of incubation in the presence of 25 μM ALLN (a non specific proteasomal inhibitor) and 10 μM MG-132 (Mancini, R, et al., 2003, J Biol Chem, 278, 46895-905). Cell lysates were prepared and were subjected to Western blot analysis using anti-glucocerebrosidase, anti-p53 and anti-erk antibodies. p53 was used as a positive control since it is subjected to ERAD and is stabilized using proteasomal inhibitors (Maki et al., 1996). As is shown in FIGS. 3a-b, glucocerebrosidase levels in cells of the affected GD brothers exhibited a significant stabilization following incubation with the proteasome inhibitors. Thus, in cells of the severely affected brother (individual II4 as depicted in FIG. 1) incubation with the proteasome inhibitors resulted in an increase in glucocerebrosidase levels from 38% to 86% of normal. A significant, but less drastic stabilization of glucocerebrosidase was observed in cells of the mildly affected brother (individual II2 as depicted in FIG. 1) which was from 43% to 63% of normal. On the other hand, the glucocerebrosidase levels in cells derived from a normal, unaffected individual, remained constant (FIGS. 3a-b).

Stabilization of mutant glucocerebrosidase by proteasome inhibitors correlates with disease severity—FIGS. 10a-g present similar analysis performed on additional cells from GD patients. Fibroblast cells were grown in the presence of the proteasomal inhibitors ALLN and MG-132 and were subjected to Western blot analysis. As is shown in FIG. 10a, while the level of glucocerebrosidase from control cells (WT) was not affected by proteasomal inhibitors, mutant glucocerebrosidase variants were stabilized in almost all GD patients that were tested. P53, which undergoes proteasomal degradation and can be stabilized by proteasomal inhibitors (Maki, C. G., et al., 1996) was used as a control. In addition, as is further shown in FIG. 10d, there was larger glucocerebrosidase stabilization in patients with neuronopathic GD (from 2.2 to 3.8 fold increase in glucocerebrosidase level) as compared to mild type 1 patients (from 0.9 to 1.4 fold increase in glucocerebrosidase levels). In patients with severe type 1 disease (GD subjects 9 and 11), the level of stabilization was higher (1.5-1.7 fold increase) than that presented by mild type 1 patients (subjects 6, 8, 10, fold increase of 0.9-1.5; FIG. 10d). These results indicate that the decrease in glucocerebrosidase levels in GD patients is due to proteasomal degradation and presents a correlation between the level of ER degradation and disease severity.

These findings corroborate well with the Endo-H sensitivity of glucocerebrosidase from GD patients. These results strongly suggest that in the severely affected cases most of the glucocerebrosidase retains in the ER and undergoes ERAD process, whereas in the mildly affected cases less glucocerebrosidase undergoes ERAD and some of it skips the process. Thus, these findings suggest that all mutant glucocerebrosidase variants undergo the ERAD process to different extents. These findings further suggest that stabilization of glucocerebrosidase mutant variants can improve the prognosis of GD patients.

### Example 3

### Recombinant Gaucher Disease Mutant Variants of Glucocerebrosidases are Subject to ERAD

To test the hypothesis that all recombinant glucocerebrosidase variants undergo ERAD, the present inventors transfected HeLa cells with plasmids expressing normal or mutated glucocerebrosidase variants, and determined the presence of immature glucocerebrosidase, as follows.

Preparation of recombinant GD Variants of glucocerebrosidase in plasmids—The following mutations were introduced into a glucocerebrosidase expressing plasmid: K157Q, D140H and E326K (which are present in individuals of the GD family depicted in FIG. 1); L444P, a severe mutation which when inherited in the homozygous form results in type 3 GD (Dahl et al., 1990; Tsuji et al., 1987); P415R, a very severe mutation associated with type 2 Gaucher disease (Wigderson et al., 1989); D409H, a mutation that leads to pseudo GD in homozygocity, characterized by oculomotor apraxia and a progressive cardiac valve defect with minimal organomegaly (Eyal et al., 1990; The ophilus et al., 1989). Previous results indicated that this mutation leads to reduction in glucocerebrosidase stability (Pasmanik-Chor et al., 1996); G202R, a mutation that was found in homozygocity in patients presenting type 2 Gaucher disease and was described as inhibiting transport of glucocerebrosidase from the ER to the lysosomes (Zimmer et al., 1999). All mutant cDNAs were coupled to a myc-tag in the pcDNA4 myc his-B expression vector (Invitrogen Life-technologies).

Glucocerebrosidase mutant variants exhibit Endo-H sensitivity—HeLa cell were transfected with the plasmids encoding the different myc-tagged glucocerebrosidase variants (WT, K157Q, D140H, D140H-E326K, G202R and N370S). Twenty-four hours after transfection, cell lysates were prepared and further subjected to Endo-H treatment followed by Western blot analysis. As is shown in FIG. 4, the normal myc-glucocerebrosidase was Endo-H resistant, indicating that this system is adequate for studying glucocerebrosidase processing. On the other hand, all mutated forms of glucocerebrosidase that were tested thus far showed Endo-H sensitivity.

These results demonstrate that the recombinant glucocerebrosidase variants K157Q, D140H, D140H-E326K, G202R and N370S are present in an immature form which does not reach the mid-Golgi apparatus. These results therefore suggest the retention of such recombinant glucocerebrosidase mutants in the ER and their possible association with the ER sugar specific chaperone, calnexin.

Calnexin is a type I transmembrane protein, localized in the ER, that associate selectively with incompletely folded glycoproteins containing monoglycosylated N-linked oligosaccharides (Wada et al., 1991). It recognizes the highly mannosylated sugar on ER proteins. Proteins that are degraded by the ER associated proteasome pathway get ubiquitinated.

Glucocerebrosidase mutant variants co-immunoprecipitated with ubiquitin—To substantiate the hypothesis that mutant glucocerebrosidase variants are subject to ERAD via ubiquitination and degradation by the proteasome machinery, lysates of HeLa cells transfected with the different glucocerebrosidase mutant variants (WT, K157Q, D140H, E326K, D140H-E326K, G202R, N370S, D409H, P415R and L444P) were immunoprecipitated with an anti-myc antibody and the precipitates were electrophoresed through 10% SDS-PAGE and were analyzed by Western blot using anti-myc or anti-ubiquitin antibodies. The mutant forms of glucocerebrosidase were co-immunopercipitated with anti ubiquitin antibodies (data not shown) arguing that the mutant glucocerebrosidase forms are subjected to ERAD and as a step in this process they are linked to ubiquitin.

Altogether, these results demonstrate that GD recombinant mutant variants of glucocerebrosidase are subject to ERAD and ubiquitination via the proteasome machinery.

### Example 4

### GD Mutant Glucocerebrosidase Variants Co-Localize and Interact with Calnexin

Experimental Results

ER retention of glucocerebrosidase in GD cells—To test glucocerebrosidase localization, indirect immunofluorescence was performed. As shown in FIGS. 8a-u, in normal cells, glucocerebrosidase accumulated in punctate lysosomal structures, as presented by co-localization with lysotracker (FIGS. 9a-f). Only a negligible fraction of glucocerebrosidase was co-localized with calnexin, an ER marker. On the other hand, all mutant glucocerebrosidase variants demonstrated diverse levels of co-localization with calnexin. Levels of co-localization with calnexin correlated well with endo-H sensitivity and disease severity. In cells from severe GD patients there was almost complete co-localization of glucocerebrosidase with calnexin, indicating that most of the protein was retained in the ER and did not reach the lysosomes. In cells from mildly affected patients part of the protein showed a reticular accumulation in the calnexin positive ER, while it also appeared in punctate lysosomal structures.

To ensure that there is no defect in glucocerebrosidase sorting in GD patients, cells derived from normal, mildly affected and severely affected patients, transiently expressing myc tagged WT glucocerebrosidase were subjected to indirect immunofluorescence. The results demonstrated that in all the tested cells, glucocerebrosidase accumulated in punctuate structures and did not co-localize with calnexin in the ER (data not shown), implying that normal glucocerebrosidase can reach its target localization in GD cells though the endogenous enzyme fails to do so, and that the mis-localization of glucocerebrosidase in GD patients is due to the presence of the mutated protein and not to a sorting defect.

Recombinant myc-tagged mutated glucocerebrosidase variants are endo-H sensitive and retain in the ER—It was interesting to test whether recombinant glucocerebrosidase variants behave similarly to their endogenous counterparts. To do that, cell lysates prepared from HeLa cells, transiently transfected with normal or mutated myc tagged glucocerebrosidase variants, were subjected to endo-H treatment and Western blot analysis using anti-myc antibody. The results (FIG. 11) showed that a major fraction of the normal myc-glucocerebrosidase was endo-H resistant. However, all tested mutants were endo-H sensitive. No difference in endo-H sensitivity between the different mutated forms was detected, most probably, due to the over-expression of the recombinant proteins, which exhausted the ER, increased the ER stress and therefore—the ERAD process.

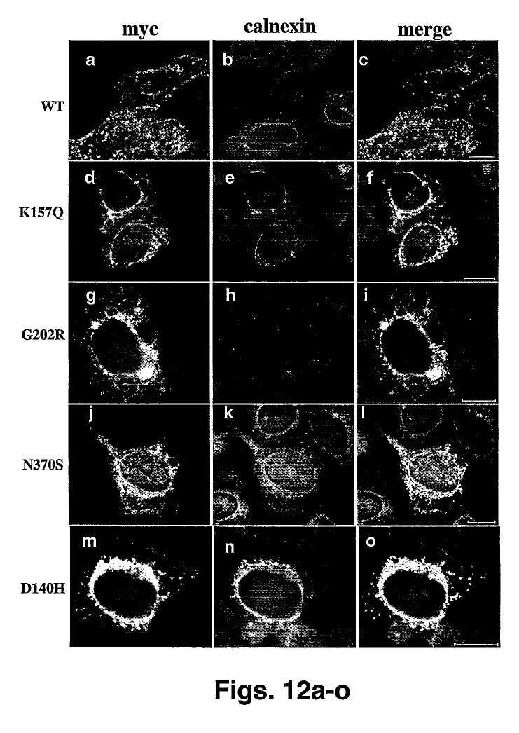

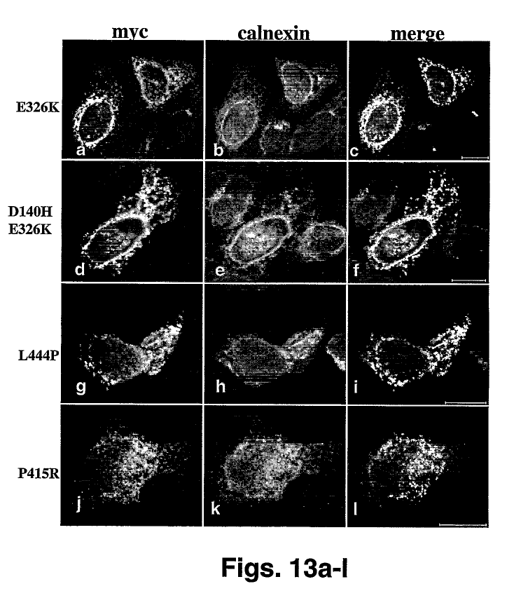

To test the hypothesis that the myc-tagged mutated glucocerebrosidase forms are retained in the ER and therefore are endo-H sensitive, their intracellular localization was tested. As presented in FIGS. 12a-c, most over-expressed normal (WT) glucocerebrosidase was localized in punctate lysosomal structures, with no calnexin colocalization. On the other hand, all mutants presented major co-localization with calnexin, indicating that most of the mutated recombinant proteins were retained in the ER (FIGS. 12d-o and 13a-l).

Mutated glucocerebrosidase variants interact with calnexin—Since all tested recombinant mutant glucocerebrosidase forms were endo-H sensitive and were retained in the ER, their possible association with the ER sugar specific chaperone calnexin was tested. Cainexin is a type I transmembrane protein localized in the ER, that associates selectively with incompletely folded glycoproteins containing monoglycosylated N-linked oligosaccharides (Wada, I., et al., 1991). It participates in ERAD of some misfolded glycoproteins and was shown to transiently interact with a large number of newly synthesized transmembrane and secretory glycoproteins, from which it dissociates after they attain a native conformation (Pind, S., et al., 1994; David, V., et al., 1993; Degen, E., et al., 1992; Ou, W. J., et al., 1993). If the protein is mis-folded, calnexin fails to dissociate from it and seems to lead the mutant protein to ERAD (Pind, S., et al., 1994; Jackson, M. R., et al., 1994; Rajagopalan, S., et al., 1994). Lysates of cells, transfected with plasmids expressing different mutant myc tagged glucocerebrosidase variants were subjected to immunoprecipitation with anti myc antibody and Western blot analysis with anti-calnexin antibodies. As is shown in FIGS. 14a-c a small fraction of the WT myc-tagged glucocerebrosidase was capable of binding calnexin. This fraction reflects part of the newly synthesized glucocerebrosidase which is present in the ER. On the other hand, as is further shown in FIGS. 14a-c, mutant glucocerebrosidase variants exhibited a significantly higher binding capacity to calnexin, ranging between 1.7 folds (for the 370S variant) to 15.3 fold (for the K157Q variant) of that of WT glucocerebrosidase. These results indicate that variant forms of glucocerebrosidase interact with calnexin in the ER.

The endogenous interaction between glucocerebrosidase and calnexin was further tested. For that purpose, cell lysates of an unaffected individual (subject 1), GD type 3 (subject 3) or GD type 2 (subject 13) were treated with MG-132 following which they were immunopercipitated using an anti calnexin antibody. Immunoprecipitates or whole cell lysates were subjected to Western blot analysis using anti glucocerebrosidase and anti calnexin antibodies. The results presented in FIGS. 14d-i showed that mutant glucocerebrosidase interacted with calnexin, while there was no detectable interaction with the normal protein. The level of calnexin bound glucocerebrosidase was higher in MG-132 treated cells. The fact that WT myc tagged glucocerebrosidase, overexpressed in cells, interacted with calnexin while endogenous normal glucocerebrosidase did not, implies that there is some retention of normal overexpressed protein in the ER, as presented by its marginal endo-H sensitivity (see FIG. 11).

Analysis and Discussion

More then 200 mutations in the glucocerebrosidase gene have been associated with Gaucher disease. In general, they account for the heterogeneity of the disease. However the heterogeneity among patients with the same phenotype could not be explained (Grabowski, G. A., 2004).

The findings of the present study demonstrate variable levels of ER retention and degradation leading to decreased mutant glucocerebrosidase levels among Gaucher disease patients. The decrease in glucocerebrosidase level could partially be stabilized by proteasomal inhibitors, implicating that at list part of the decrease in protein levels is due to proteasomal degradation. The present findings show that the mutant proteins are bound to calnexin, a chaperon localized in the ER, that associates selectively with incompletely folded glycoproteins and participates in ERAD of some misfolded glycoproteins (Pind S, et al., 1994; Ou, W. J., et al., 1993). There was significant correlation between endo-H sensitivity, ER retention, protein degradation (Varga, K., et al., 2004) and disease severity. It is worth mentioning that this data is based on experiments performed on endogenous, native, glucocerebrosidase forms. Though normal recombinant glucocerebrosidase behaved very similar to the endogenous counterpart and only a small fraction of it was endo-H sensitive and bound calnexin, all mutant recombinant forms were retained in the ER. There are documented cases, in which maturation of a normal recombinant protein differs from that of the endogenous protein. This could be due to lack of binding partners, whose association with the expressed protein may be required for proper maturation and/or trafficking. Thus, it has been shown that a significant fraction of normal recombinant CFTR is retained within the ER and is subjected to ERAD (Cheng, S. H., et al., 1990; Ward, C. L. and Kopito, R. R., 1994; Ward, C. L., et al., 1995), while endogenous CFTR exits from the ER, with no retention there (Varga, K., et al., 2004).

The results of the present study suggest that the ERAD process of mutant glucocerebrosidase forms plays a significant role in determining the disease heterogeneity. Patients with the same genotype may show different disease severities due to different fidelities of their quality control. The level of ER retention and concomitant decrease in protein level determine disease severity. To date, there are no direct means to correlate between disease severity and any biochemical/molecular test. These findings suggest the use of levels of immature glucocerebrosidase in GD patients, namely endo-H sensitivity, as a tool to implicate disease severity and/or prognosis.

The importance of ER quality control in general and the ERAD process in particular, has been indicated in a large spectrum of diseases (Tsai, B., et al., 2003; Sitia, R. and Braakman, I., 2003; Kostova, Z. and Wolf, D. H., 2003). The Cystic Fibrosis ΔF508—CFTR mutated protein does not reach its plasma membrane localization in lung epithelial cells due to its slow or inefficient folding in the ER and excessive degradation (Denning, G. M., et al., 1992; Gelman, M. S, and Kopito, R. R., 2003; Xiong, X., et al., 1999). The intracellular aggregation of the mis-folded mutant rhodopsin leads to Retinitis Pigmentosa. This aggregation results from the retrotranslocation of the misfolded protein by the ERAD machinery, but there is inefficient degradation of this misfolded protein due to saturation of the normal proteolytic machinery (Saliba, R. S., et al., 2002).

It seems that the ERAD process is the mechanism underlying disorders associated with mutant proteins that are processed in the ER, including lysosomal disorders. The possibility that mutant lysosomal enzymes are retained in the ER and undergo ERAD has been suggested for β-hexosaminidase A in chronic adult GM2 gangliosidoses β-galactosidase (Tropak, M. B., et al., 2004), mutated in GM1 gangliosidoses and Morquio B disease (Zhang, S., et al., 2000), as well as c-galactosidase, whose impaired activity causes Fabry disease (Asano, N., et al., 2000).

Studying the ERAD machinery is beginning to provide significant medical insights. Understanding the involvement of this process in pathogenesis is opening a novel approach of pharmacological intervention (Perlmutter, D. H., 2002; Welch, W. J. and Howard, M., 2000). Two main strategies are being pursued to obtain functional rescue: the first involves the development of substances that favor correct folding of mutant proteins and consequently allow them to pass the quality control machinery. The second strategy involves release of fractions of these mis-folded proteins from the ER by preventing their interactions with the quality control components. In both cases, the released mutant proteins, which may have residual activity, reach their normal destination. It has already been shown that low temperature or nonspecific chemical chaperones (such as glycerol) release fraction of ΔF508-CFTR protein to the plasma membrane, where it is active (Denning, G. M., et al., 1992; Brown, C. R., et al., 1997).

Recent studies are focused on using more specific agonists as pharmacological chaperones to rescue proteins with medical relevance from their retention compartments (Perlmutter, D. H., 2002; Welch, W. J. and Howard, M., 2000; Morello, J. P., et al., 2000). Thus, it has been shown, in the case of ΔF508-CFTR mutant that A₁ adenosine receptor antagonist 8-cyclophenyl-1,2-diproylxantthine (CPX) as well as benzo(c)quinolizinium drugs MBP-07 and MBP-91 lead to restoration of the plasma membrane localization of the AF508-CFTR, in vitro, most likely due to stabilizing correct folding of the mutant protein by involving specific binding sites (Dormer, R. L., et al., 2001; Zeitlin, P. L., 2000, Respiration, 67, 351-7; Zeitlin, P. L., 2000, Kidney Int, 57, 832-7). Similarly, the rhodopsin P23H mutant, causing Retinitis Pigmentosa, could be rescued by the retinal derivate 11-cis-7-ring retinal (Noorwez, S. M., et al., 2003). This approach has been applied already in lysosomal enzymes. Recent study has demonstrated that sub-inhibitory doses of the competitive inhibitor of the α-galactosidase A, DJG (Yam, G. H., et al., 2005), releases Fabry mutants from the ER chaperone BIP, which are transported to the lysosomes, leading to clearance of the lysosomal storage. In the case of GD, it has been shown that lysosomal levels and activity of the F213I and the cellular activity of the N370S glucocerebrosidase mutants are increased by treatment with the glucocerebrosidase inhibitors NOV and NN-DNJ, respectively (Sawkar, A. R., et al., 2002; Ogawa, S., et al., 2002).

Since only a 1-5% of normal intracellular glucocerebrosidase activity is required to correct the metabolic defect in GD cells (Desnick, R. J., 2004), specific small-molecule ligands that act as pharmacological chaperones and enhance the mis-folded mutant glucocerebrosidase variants trafficking to the lysosomes will improve their residual activity and will serve as a basis for therapy in the treatment of this disease.

To Summarize, these results strongly indicate a direct correlation between Gaucher disease severity and glucocerebrosidase level, endo-H sensitivity, ER localization, binding to calnexin and proteasomal degradation. This is also true for patients with the same genotype who present different disease severity. Therefore, these results suggest to use levels of immature glucocerebrosidase in GD patients as a tool to implicate disease severity.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Sandhoff, K. Harzer, K. and Furst, W. (1995) Sphingolipid activator proteins. In Scriber, S., Beaudet, A., Sly, W., Valle, D. (ed.), The Metabolic and Molecular Basis of Inherited Disease. 7 ed. McGrew Hill, pp. 2427-2441.
2. Christomanou, H., Chabas, A., Pampols, T. and Guardiola, A. (1989) Activator protein deficient Gaucher's disease. A second patient with the newly identified lipid storage disorder. Klin Wochenschr, 67, 999-1003.
3. Beutler, E. and Grabowski, G. A. (1995) Gaucher Disease. In Scriver, C. R., Beaudet, A. L., Sly, W. S, and Valle, D. (eds.), The Metabolic and Molecular Bases of Inherited Diseases. Seven ed. McGraw Hill, Vol. II, pp. 2641-2663.
4. Erickson, A. H., Ginns, E. I. and Barranger, J. A. (1985) Biosynthesis of the lysosomal enzyme glucocerebrosidase. J Biol Chem, 260(26), 14319-14324.
5. Glickman, J. N. and Komfeld, S. (1993) Mannose 6-phosphate-independent targeting of lysosomal enzymes in I-cell disease B lymphoblasts. J Cell Biol, 123, 99-108.
6. Zimmer, K. P., le Coutre, P., Aerts, H. M., Harzer, K., Fukuda, M., O'Brien, J. S, and Naim, H. Y. (1999) Intracellular transport of acid beta-glucosidase and lysosome-associated membrane proteins is affected in Gaucher's disease (G202R mutation). J Pathol, 188, 407-14.
7. Sawkar, A. R., Cheng, W. C., Beutler, E., Wong, C. H., Balch, W. E. and Kelly, J. W. (2002) Chemical chaperones increase the cellular activity of N370S beta -glucosidase: a therapeutic strategy for Gaucher disease. Proc Natl Acad Sci USA, 99, 15428-33.
8. Ogawa, S., Matsunaga, Y. K. and Suzuki, Y. (2002) Chemical modification of the beta-glucocerebrosidase inhibitor N-octyl-beta-valienamine: synthesis and biological evaluation of 4-epimeric and 4-O-(beta-D-galactopyranosyl) derivatives. Bioorg Med Chem, 10, 1967-72.
9. Lin, H., Sugimoto, Y., Ohsaki, Y., Ninomiya, H., Oka, A., Taniguchi, M., Ida, H., Eto, Y., Ogawa, S., Matsuzaki, Y. et al. (2004) N-octyl-beta-valienamine up-regulates activity of F213I mutant beta-glucosidase in cultured cells: a potential chemical chaperone therapy for Gaucher disease. Biochim Biophys Acta, 1689, 219-28.
10. Tsai, B., Ye, Y. and Rapoport, T. A. (2002) Retro-translocation of proteins from the endoplasmic reticulum into the cytosol. Nat Rev Mol Cell Biol, 3, 246-55.
11. Hammond, C. and Helenius, A. (1995) Quality control in the secretory pathway. Curr Opin Cell Biol, 7, 523-9.
12. Sitia, R. and Braakman, I. (2003) Quality control in the endoplasmic reticulum protein factory. Nature, 426, 891-4.
13. Brodsky, J. L. and McCracken, A. A. (1999) ER protein quality control and proteasome-mediated protein degradation. Semin Cell Dev Biol, 10, 507-13.
14. Jarosch, E., Geiss-Friedlander, R., Meusser, B., Walter, J. and Sommer, T. (2002) Protein dislocation from the endoplasmic reticulum—pulling out the suspect. Traffic, 3, 530-6.
15. Tropak, M. B., Reid, S. P., Guiral, M., Withers, S. G. and Mahuran, D. (2004) Pharmacological enhancement of beta-hexosaminidase activity in fibroblasts from adult Tay-Sachs and Sandhoff Patients. J Biol Chem, 279, 13478-87.
16. Zhang, S., Bagshaw, R., Hilson, W., Oho, Y., Hinek, A., Clarke, J. T. and Callahan, J. W. (2000) Characterization of beta-galactosidase mutations Asp332-->Asn and Arg148-->Ser, and a polymorphism, Ser532—>Gly, in a case of GM1 gangliosidosis. Biochem J, 348 Pt 3, 621-32.
17. Asano, N., Ishii, S., Kizu, H., Ikeda, K., Yasuda, K., Kato, A., Martin, O. R. and Fan, J. Q. (2000) In vitro inhibition and intracellular enhancement of lysosomal alpha-galactosidase A activity in Fabry lymphoblasts by 1-deoxygalactonojirimycin and its derivatives. Eur J Biochem, 267, 4179-86.
18. Yam, G. H., Zuber, C. and Roth, J. (2005) A synthetic chaperone corrects the trafficking defect and disease phenotype in a protein misfolding disorder. Faseb J, 19, 12-8.
19. Bradford, M. M. (1976) A rapid and sensitive method for the quantitation of microgram quantities of protein utilizing the principle of protein-dye binding. Anal Biochem, 72, 248-54.
20. Plummer, T. H., Jr., Elder, J. H., Alexander, S., Phelan, A. W. and Tarentino, A. L. (1984) Demonstration of peptide:N-glycosidase F activity in endo-beta-N-acetylglucosaminidase F preparations. J Biol Chem, 259, 10700-4.
21. Trimble, R. B. and Tarentino, A. L. (1991) Identification of distinct endoglycosidase (endo) activities in Flavobacterium meningosepticum: endo F1, endo F2, and endo F3. Endo F1 and endo H hydrolyze only high mannose and hybrid glycans. J Biol Chem, 266, 1646-51.
22. Maley, F., Trimble, R. B., Tarentino, A. L. and Plummer, T. H., Jr. (1989) Characterization of glycoproteins and their associated oligosaccharides through the use of endoglycosidases. Anal Biochem, 180, 195-204.
23. Pasmanik-Chor, M., Madar-Shapiro, L., Stein, E. O., Aerts, H., Gatt, S, and Horowitz, M. (1997) Expression of mutated glucocerebrosidase alleles in human cells. Hum Mol Genet, 6, 887-95.
24. Tsai, B. and Rapoport, T. A. (2002) Unfolded cholera toxin is transferred to the ER membrane and released from protein disulfide isomerase upon oxidation by Erol. J Cell Biol, 159, 207-16.
25. Bonifacino, J. S. and Weissman, A. M. (1998) Ubiquitin and the control of protein fate in the secretory and endocytic pathways. Annu Rev Cell Dev Biol, 14, 19-57.
26. Maki, C. G., Huibregtse, J. M. and Howley, P. M. (1996) In vivo ubiquitination and proteasome-mediated degradation of p53(1). Cancer Res, 56, 2649-54.
27. Mancini, R., Aebi, M. and Helenius, A. (2003) Multiple endoplasmic reticulum-associated pathways degrade mutant yeast carboxypeptidase Y in mammalian cells. J Biol Chem, 278, 46895-905.
28. Wada, I., Rindress, D., Cameron, P. H., Ou, W. J., Doherty, J. J., 2nd, Louvard, D., Bell, A. W., Dignard, D., Thomas, D. Y. and Bergeron, J. J. (1991) SSR alpha and associated calnexin are major calcium binding proteins of the endoplasmic reticulum membrane. J Biol Chem, 266, 19599-610.
29. Pind, S., Riordan, J. R. and Williams, D. B. (1994) Participation of the endoplasmic reticulum chaperone calnexin (p88, IP90) in the biogenesis of the cystic fibrosis transmembrane conductance regulator. J Biol Chem, 269, 12784-8.
30. David, V., Hochstenbach, F., Rajagopalan, S. and Brenner, M. B. (1993) Interaction with newly synthesized and retained proteins in the endoplasmic reticulum suggests a chaperone function for human integral membrane protein IP90 (calnexin). J Biol Chem, 268, 9585-92.
31. Degen, E., Cohen-Doyle, M. F. and Williams, D. B. (1992) Efficient dissociation of the p88 chaperone from major histocompatibility complex class I molecules requires both beta 2-microglobulin and peptide. J Exp Med, 175, 1653-61.
32. Ou, W. J., Cameron, P. H., Thomas, D. Y. and Bergeron, J. J. (1993) Association of folding intermediates of glycoproteins with calnexin during protein maturation. Nature, 364, 771-6.
33. Jackson, M. R., Cohen-Doyle, M. F., Peterson, P. A. and Williams, D. B. (1994) Regulation of MHC class I transport by the molecular chaperone, calnexin (p 88, IP90). Science, 263, 384-7.
34. Rajagopalan, S., Xu, Y. and Brenner, M. B. (1994) Retention of unassembled components of integral membrane proteins by calnexin. Science, 263, 387-90.
35. Grabowski, G. A. (2004) Gaucher disease: lessons from a decade of therapy. J Pediatr, 144, S15-9.
36. Varga, K., Jurkuvenaite, A., Wakefield, J., Hong, J. S., Guimbellot, J. S., Venglarik, C. J., Niraj, A., Mazur, M., Sorscher, E. J., Collawn, J. F. et al. (2004) Efficient intracellular processing of the endogenous cystic fibrosis transmembrane conductance regulator in epithelial cell lines. J Biol Chem, 279, 22578-84.
37. Cheng, S. H., Gregory, R. J., Marshall, J., Paul, S., Souza, D. W., White, G. A., O'Riordan, C. R. and Smith, A. E. (1990) Defective intracellular transport and processing of CFTR is the molecular basis of most cystic fibrosis. Cell, 63, 827-34.
38. Ward, C. L. and Kopito, R. R. (1994) Intracellular turnover of cystic fibrosis transmembrane conductance regulator. Inefficient processing and rapid degradation of wild-type and mutant proteins. J Biol Chem, 269, 25710-8.
39. Ward, C. L., Omura, S, and Kopito, R. R. (1995) Degradation of CFTR by the ubiquitin-proteasome pathway. Cell, 83, 121-7.
40. Kostova, Z. and Wolf, D. H. (2003) For whom the bell tolls: protein quality control of the endoplasmic reticulum and the ubiquitin-proteasome connection. Embo J, 22, 2309-17.
41. Denning, G. M., Anderson, M. P., Amara, J. F., Marshall, J., Smith, A. E. and Welsh, M. J. (1992) Processing of mutant cystic fibrosis transmembrane conductance regulator is temperature-sensitive. Nature, 358, 761-4.
42. Gelman, M. S. and Kopito, R. R. (2003) Cystic fibrosis: premature degradation of mutant proteins as a molecular disease mechanism. Methods Mol Biol, 232, 27-37.
43. Xiong, X., Chong, E. and Skach, W. R. (1999) Evidence that endoplasmic reticulum (ER)-associated degradation of cystic fibrosis transmembrane conductance regulator is linked to retrograde translocation from the ER membrane. J Biol Chem, 274, 2616-24.
44. Saliba, R. S., Munro, P. M., Luthert, P. J. and Cheetham, M. E. (2002) The cellular fate of mutant rhodopsin: quality control, degradation and aggresome formation. J Cell Sci, 115, 2907-18.
45. Perlmutter, D. H. (2002) Chemical chaperones: a pharmacological strategy for disorders of protein folding and trafficking. Pediatr Res, 52, 832-6.
46. Welch, W. J. and Howard, M. (2000) Antagonists to the rescue. J Clin Invest, 105, 853-4.
47. Brown, C. R., Hong-Brown, L. Q. and Welch, W. J. (1997) Strategies for correcting the delta F508 CFTR protein-folding defect. J Bioenerg Biomembr, 29, 491-502.
48. Morello, J. P., Petaja-Repo, U. E., Bichet, D. G. and Bouvier, M. (2000) Pharmacological chaperones: a new twist on receptor folding. Trends Pharmacol Sci, 21, 466-9.
49. Dormer, R. L., Derand, R., McNeilly, C. M., Mettey, Y., Bulteau-Pignoux, L., Metaye, T., Vierfond, J. M., Gray, M. A., Galietta, L. J., Morris, M. R. et al. (2001) Correction of delF508-CFTR activity with benzo(c)quinolizinium compounds through facilitation of its processing in cystic fibrosis airway cells. J Cell Sci, 114, 4073-81.
50. Zeitlin, P. L. (2000) Future pharmacological treatment of cystic fibrosis. Respiration, 67, 351-7.
51. Zeitlin, P. L. (2000) Pharmacologic restoration of delta F508 CFTR-mediated chloride current. Kidney Int, 57, 832-7.
52. Noorwez, S. M., Kuksa, V., Imanishi, Y., Zhu, L., Filipek, S., Palczewski, K. and Kaushal, S. (2003) Pharmacological chaperone-mediated in vivo folding and stabilization of the P23H-opsin mutant associated with autosomal dominant retinitis pigmentosa. J Biol Chem, 278, 14442-50.
53. Desnick, R. J. (2004) Enzyme replacement and enhancement therapies for lysosomal diseases. J Inherit Metab Dis, 27, 385-410.
54. Beutler, E. (1995). Gaucher disease, a paradigm for single gene defects. Experientia 51, 196-197.
55. Beutler, E., Gelbart, T., Kuhl, W., Sorge, J., and West, C. (1991). Identification of the second commonjewish Gaucher disease mutation makes possible population-based screening for the heterozygous state. Proc Nat Ac˜Sci USA 88, 10544-10547.
56. Brady, R. 0., Kafner, J. N., and Shapiro, D. (1965). Metabolism of glucocerebrosidase II. Evidence of an enzymatic deficiency in Gaucher's disease. Biochem Biophys Res commun 18(2), 221-225.
57. Cormand, B., Diaz, A., Grinberg, D., Chabas, A., and Vilageliu, L. (2000). A new gene-pseudogene fusion allele due to a recombination in intron 2 of the glucocerebrosidase gene causes gaucher disease [In Process Citation]. Blood Cells Mol Dis 26, 409-416.
58. Dahl, N., Lagerstrom, M., Erikson, A., and Pettersson, U. (1990). Gaucher disease type III (Norrbottnian type) is caused by a single mutation in exon 10 of the glucocerebrosidase gene. Am J Hum Genet. 47, 275-278.
59. Eyal, N., Firon, N., Wilder, S., Kolodny, H., and Horowitz, M. (1991). Three new base pair changes in a fami] with Gaucher disease. Hum Genet. 87, 328-332.
60. Eyal, N., Wilder, S., and Horowitz, M. (1990). Prevalent and rare mutations among Gaucher patients. Gene 96 277-283.
61. Gelman, M. S., Kannegaard, E. S., and Kopito, R. R. (2002). A principal role for the proteasome in endoplasmic reticulum-associated degradation of misfolded intracellular cystic fibrosis transmembrane conductance regulator. J Biol Chem 277, 11709-11714.
62. Grace, M. E., Ashton-Prolla, P., Pastores, G. M., Soni, A., and Desnick, R. J. (1999). Non-pseudogene-derive< complex acid beta-glucosidase mutations causing mild type 1 and severe type 2 gaucher disease. J Clin Invest 103, 817-823.
63. Gravel R. A. et al., 1995. The GM2 gangliosidoses. In: Scriver C R, Beaudet A L, Sly W S, Valle D, eds. The Metabolic and Molecular Bases of Inherited Disease. New York: McGraw-Hill, 1995: 28392882.
64. He, G. S., and Grabowski, G. A. (1992). Gaucher disease: A G+1----A+1 IVS2 splice donor site mutation causing exon 2 skipping in the acid beta-glucosidase mRNA. Am J Hum Genet. 51, 810-820.
65. Horowitz, M., Pasmanik-Chor, M., Borochowitz, Z., and al, e. (1998). Prevalence of glucocerebrosidase mutations in the Israeli Jewish population. Hum Mut. 2: 240-244.
66. Latham, T., Grabowski, G. A., Theophilus, B. D., and Smith, F. I. (1990). Complex alleles of the acid betaglucosidase gene in Gaucher disease. Am J Hum Genet. 47, 79-86.
67. Lu, Y., Xiong, x., Helm, A., Kirnani, K., Bragin, A., and Skach, W. R. (1998). Co- and posttranslational translocation mechanisms direct cystic fibrosis transmembrane conductance regulator N terminus transmembrane assembly. J Biol Chem 273, 568-576.
68. Madar-Shapiro, L., Pasmanik-Chor, M., Dinur, T., Dagan, A., Gatt, S., and Horowitz, M. (1999). Intracellular degradation of fluorescent glycolipids by lysosomal enzymes and their activators [In Process Citation]. J Inher Metab Dis 22, 623-637.
69. Pasmanik-Chor, M., Laadan, S., Elroy-Stein, 0., Zimran, A., Abrahamov, A., Gatt, S., and Horowitz, M. (1996). The Glucocerebrosidase D409H Mutation in Gaucher Disease. Biocem Molec Med 59, 125-133.
70. Sinclair, G., Choy, F. Y., and Humphries, L. (1998). A novel complex allele and two new point mutations in type 2 (acute neuronopathic) Gaucher disease. Blood Cells Mol Dis 24, 420-427.
71. Theophilus, B. D., Latham, T., Grabowski, G. A., and Smith, F. I. (1989). Comparison of RNase A, a chemica cleavage and GC-clamped denaturing gradient gel electrophoresis for the detection of mutations in exon 9 of the human acid beta-glucosidase gene. Nucleic Acids Res 17, 7707-7722.
72. Tsai, B., and Rapoport, T. A. (2002). Unfolded cholera toxin is transferred to the ER membrane and released from protein disulfide isomerase upon oxidation by Ero 1. J Cell Biol 159, 207-216.
73. Tsuji, S., Choudary, P. V., Martin, B. M., Stubblefield, B. K., Mayor, J. A., Barranger, J. A., and Ginns, E. I. (1987). A mutation in the human glucocerebrosidase gene in neuronopathic Gaucher's disease. N Engl J Med 316, 570-575.
74. Tsuji, S., Martin, B. M., Barranger, J. A., Stubblefield, B. K., LaMarca, M. E., and Ginns, E. I. (1988). Genetic heterogeneity in type 1 Gaucher disease: Multiple genotypes in Ashkenazic and non-Ashkenazic individuals. Proc Natl Acad Sci USA 85, 2349-2352.
75. Wigderson, M., Firon, N., Horowitz, Z., Wilder, S., Frishberg, Y., Reiner, 0., and Horowitz, M. (1989). Characterization of mutations in Gaucher patients by cDNA cloning. Am J Hum Genet. 44, 365-377.

1-34. (canceled)

35. A method of treating a Gaucher disease in a subject, the method comprising administering to the subject an agent capable of inhibiting proteasomal degradation of glucocerebrosidase thereby treating the Gaucher disease in the subject.

36. A method of treating a Gaucher disease in a subject, the method comprising administering to the subject an agent capable of elevating a level of mis-folded yet active glucocerebrosidase in cell lysosomes, thereby treating the Gaucher disease in the subject.

37. The method of claim 35, wherein said subject suffers from a type 1, type 2, type 3 or pesudo Gaucher disease.

38. The method of claim 35, wherein said agent is a proteasome inhibitor.

39. The method of claim 38, wherein said proteasome inhibitor is N-acetyl-leucinyl-leucinyl-norleucinal (ALLN), MG-132, MLN519, benzyloxycarbonyl-isoleucyl-glutamyl(O-tert-butyl)-alanyl-leucinal (PSI) and/or PS-341.

40. The method of claim 35, wherein said agent is formulated for systemic administration.

41. The method of claim 36, wherein said agent is a small molecule.

42. The method of claim 36, wherein said mis-folded yet active glucocerebrosidase includes at least 4 mannose molecules attached to said glucocerebrosidase.

43. A method of identifying an agent capable of treating a Gaucher disease, the method comprising:
(a) exposing cells expressing an ER-retained glucocerebrosidase to a plurality of molecules; and
(b) identifying at least one molecule from said plurality of molecules capable of elevating a level of active glucocerebrosidase in lysosomes of said cells, said at least one molecule being the agent suitable for treating the Gaucher disease.

44. The method of claim 43, wherein said ER-retained glucocerebrosidase is encoded by a mutated glucocerebrosidase.

45. The method of claim 44, wherein said mutated glucocerebrosidase comprises a mutation selected from the group consisting of D409H (SEQ ID NO:3), P415R (SEQ ID NO:4), L444P (SEQ ID NO:5), D140H (SEQ ID NO:6), K157Q (SEQ ID NO:7), E326K (SEQ ID NO:8), D140H+E326K (SEQ ID NO:9), G202R (SEQ ID NO:10) and N370S (SEQ ID NO:11).

46. The method of claim 43, wherein said cells expressing said ER-retained glucocerebrosidase are of a Gaucher disease patient.

47. A method of diagnosing and/or assessing a severity of Gaucher disease in a subject in need thereof, the method comprising detecting in cells of the subject an ER-retained glucocerebrosidase, wherein a level of said ER-retained glucocerebrosidase is indicative for the severity of Gaucher disease in the subject.

48. A kit for diagnosing and/or assessing a severity of Gaucher disease in a subject, the kit comprising a packaging material packaging at least one reagent for detecting in cells of the subject a level of an ER-retained glucocerebrosidase thereby diagnosing and/or assessing the severity Gaucher disease in the subject.

49. The method of claim 47, wherein said glucocerebrosidase is set forth by SEQ ID NO:2.

50. The method of claim 47, wherein said ER-retained glucocerebrosidase includes more than 4 mannose molecules attached to said glucocerebrosidase protein.

51. The method of claim 47, wherein said detecting is effected by a biochemical analysis and/or a structural analysis.

52. The method of claim 51, wherein said biochemical analysis is effected by measuring endo-H sensitivity and/or co-precipitation with an ER-protein.

53. The method of claim 52, wherein said ER-protein is calnexin, calreticulin, ERp72, endoplamin (ERp99), ERp29, BIP (GRP78) and GRP94.

54. The method of claim 47, wherein a presence of about 15-42% of an endo-H sensitive glucocerebrosidase is indicative of a mild form of Gaucher disease in the subject.

55. The method of claim 47, wherein a presence of more than about 60% endo-H sensitive glucocerebrosidase is indicative of a severe form of Gaucher disease in the subject.

56. A method of diagnosing and/or assessing a severity of a disease associated with an abnormally folded protein in a subject the method comprising:
detecting a level of an ER-retained form of the protein in cells of the subject, said level being indicative of the severity of the disease associated with the abnormally folded protein.

57. A kit for diagnosing and/or assessing a severity of a disease associated with an abnormal folded protein in a subject, the kit comprising a packaging material packaging at least one reagent for detecting a level of an ER-retained form of the protein in cells of the subject thereby diagnosing and/or assessing a severity of the disease associated with the abnormally folded protein.

58. The method of claim 56, wherein said detecting is effected by endo-H sensitivity assay.

59. The method of claim 56, wherein said protein is a plasma membrane protein or a lysosomal protein.

60. The method of claim 59, wherein said plasma membrane protein is selected from the group consisting of CFTR and rhodopsin.

61. The method of claim 59, wherein said lysosomal protein is selected from the group consisting of glucocerebrosidase, β-hexosaminidase A, and α-galactosidase.

62. The method of claim 56, wherein said disease is selected from the group consisting of Gaucher disease, cystic fibrosis, Retinitis Pigmentosa, chronic adult GM2, GM1 gangliosidoses, Morquio B disease and Fabry disease.

63. The method of claim 58, wherein said endo-H sensitivity assay is effected using an immunological detection assay.

64. The method of claim 58, wherein said endo-H sensitivity assay is effected using a molecule capable of specifically binding a glycoprotein.
